SEQUENCE LISTING (25 sequences)

SEQ ID NO: 1 - DNA, 4596 bp, synthetic construct
  source 1..4596: mol_type = other DNA
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagaattcgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 2 - protein, 1532 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEEFDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 3 - DNA, 5439 bp, synthetic construct
  source 1..5439: mol_type = other DNA
tcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctg

SEQ ID NO: 4 - protein, 1813 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 5 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 6 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 7 - DNA, 6232 bp, synthetic construct
  source 1..6232: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccct

SEQ ID NO: 8 - protein, 2077 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 9 - DNA, 804 bp, synthetic construct
  source 1..804: mol_type = other DNA
atgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 10 - protein, 267 aa, synthetic construct
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 11 - DNA, 34070 bp, synthetic construct
  source 1..34070: mol_type = other DNA
aaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccataagctctaatcgcataatcgagctaagagcgattaatatgcttagctagcttagctaagctagatttctctattctcccacaatatatagcagctattagcttagcagtccgaatacgatcgtccacgatccataagcgtaacacatttgagtagcgactgacgggcaccggagccactcgagtggaattatcacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttgcctatataagcagagctcgtttagtgaaccgtcagatcgcctggagacgccatccacgctgttttgacctccatagaagacaccgggaccgatccagcctccgcggtggcggcatcggtacacaagtttgtacaaaaaagcaggcttcaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggcggctcaaaaagaaccgccgacggcagcgaattcgagcccaagaagaagaggaaagtcggaagcggagctactaacttcagcctgctgaagcaggctggagacgtggaggagaaccctggacctatgagcgagctgataacagaaaacatgcacatgaagctctacatggaaggtaccgtcaacaaccaccatttcaagtgtacttctgaaggcgaaggtaagccttacgaaggcacccaaacccaacgcattaaagtagtggaagggggacccctgcccttcgccttcgatattttggcaacaagcttcatgtacggaagtcatacgtttatcaatcatacgcaggggatccctgatttctggaaacagagtttcccagaggggttcacgtgggagcgagtaactacttacgaggacggcggggtactgacagccacacaagatacatctcttcaggatggctgcttgatctataatgtgaagattcgcggcgttaactttcccagtaacggccctgtcatgcagaaaaaaacgcttgggtgggaggctcacacagaaatgctctacccagcagatgggggtctggagggtagaactgctctggccctgaagctcgtgggcggcggtcacctcatttgcaactttaaaaccacctatcggtctaagaagccagcaaagaacctcaagatgcccggagtttattatgttgactacaggctcgagcggatcaaggaagctgacaaggaaacgtacgtggagcagcatgaagttgcagtagctcgctactgcgatttgccatctaagttgggccataagctgaattgaccactgagcaagaactatgaatgagatctttagacatattgcggccgctttctccgcgatccagacatgataagatacattgatgagtttggacaaaccaaaactagaatgcagtgaaaaaaatgccttatttgtgaaatttgtgatgctattgccttatttgtaaccattataagctgcaataaacaagttggcaggaaagactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaatacccccgccccgttgacgcaaatgggcaagcttgccgggtcgaggtaggcgtgtacggtgggaggcctatataagcaaccggtataatcaaacagaccagattgtctgtttgttaccggtgtttagtgaaccgggcgcgcctcatatcgcctggagacgccatccacgctgttttgacctccatagaagacaccgggaccgatccagcctccgcggtcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttggtctagagctagcgaattcgaatttaaatcggatccgcggccgcggccaccatggccccaaagaagaagcggaaggtgtctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacaaaaggccggcggccacgaagaaagctggccaggcaaaaaagaaaaaggaattcggcagtggagagggcagaggaagtctgctaacatgcggtgacgtcgaggagaatcctggcccaatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgaaacgtacgtcgagcagcacgaggtggcagtggccagatactgcgacctccctagcaaactggggcacaagcttaattaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctcgttggaccggtgccaccatgatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgaggctccggaaccggtgccaccatgtacccatacgatgttccagattacgcttcgccgaagaaaaagcgcaaggtcgaagcgtcctctgaggtggagttttcccacgagtactggatgagacatgccctgaccctggccaagagggcacgggatgagaggaaggcgcctgtgggagccgtgctggtgctgaacaatagagtgatcggcgagggctggaacagagccatcggcctgcacgacccaacagcccatgccgaaattatagccctgagacagggcggcctggtcatgcagaactacagactgattgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggaatgaatcaccgcgtcgaaattaccgagggaatcctggcagatgaatgtgccgccctgctgtgcgatttctatcggatgcctagacaggtgttcaatgctcagaagaaggcccagagctccatcaactctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctggagggagctcccaagaaaaagcgcaaggtaggtagttccctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccaccatgggagtgcaggtggaaaccatctccccaggtgacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaccggctagctctgaggtggagttttcccacgagtactggatgagacatgccctgaccctggccaagagggcacgggatgagagggaggtgcctgtgggagccgtgctggtgctgaacaatagagtgatcggcgagggctggctgagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacgagggagctcccaagaaaaagcgcaaggtaggtagttccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggatcctaagtcgacgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgacatccactttgcctttctctccacaggtgtccactcccaggtccaagtttggaagatctaccatgccaaagaggcccagacccgtgatcatgagtccaaagagaagaacacaggcagagcgcgcaatggaaacccagggcaagttgattgcagcggccctgggggttttacgggaaaaaggttacgcgggattccggatcgcagatgtgcccggtgctgcaggtgtctcgagaggagcgcagagccatcatttcccgacaaagcttgagcttctgcttgccacttttgaatggctttacgaacagatcaccgaacgcagtcgggctcgattagcgaaattgaagccagaggatgacgtcatccagcaaatgctggacgacgccgccgaatttttcctcgacgatgacttctctatcagccttgatttgattgtggctgccgaccgggatccagcgttacgcgagggtattcagcgcacggtagagaggaatcggtttgtcgtcgaggatatgtggcttggtgttctggtgagccgtggtctttcgcgtgatgatgcagaagatatcctttggttgatattcaattcggtgcgtgggcttgctgttcgtagcctatggcagaaggacaaagaacgctttgagcgtgtcaggaactcgacactcgaaattgcgcgagagcggtacgcgaaattcaagcgcgcggccaaaccctctggccgatcactctctgttcgagagggcagaggaagtcttctaacatgcggtgacgtggaggagaatcccggccctgaattcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgtgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggcagcggcgccaccaacttcagcctgctgaagcaggccggcgacgtggaggagaaccccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagtcgacaatcaacctctggattacaaaatttgtggttaactaactaaacttgtttattgcagcttataatggttacaaataaagcaatagcatcacaaatttcacaaataaagcatttttttcactgcattctagttgtggtttgtccaaactcatcaatgtatcttaccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagcccactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttatt

SEQ ID NO: 12 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 13 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 14 - DNA, 5253 bp, synthetic construct
  source 1..5253: mol_type = other DNA
tctgaggtggagttttcccacgagtactggatgagacatgccctgaccctggccaagagggcacgggatgagaggaaggcgcctgtgggagccgtgctggtgctgaacaatagagtgatcggcgagggctggaacagagccatcggcctgcacgacccaacagcccatgccgaaattatagccctgagacagggcggcctggtcatgcagaactacagactgattgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggaatgaatcaccgcgtcgaaattaccgagggaatcctggcagatgaatgtgccgccctgctgtgcgatttctatcggatgcctagacaggtgttcaatgctcagaagaaggcccagagctccatcaactctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctg

SEQ ID NO: 15 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggctgagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 16 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 1565 aa, synthetic construct; identical to SEQ ID NO: 6

SEQ ID NO: 18 - protein, 1751 aa, synthetic construct
SEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 19 - protein, 1565 aa, synthetic construct
SEVEFSHEYWMRHALTLAKRARDEREVPVGAVLVLNNRVIGEGWLRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 20 - DNA, 20 bp, synthetic construct
  source 1..20: mol_type = other DNA
gtaacggcagacttctcctc

SEQ ID NO: 21 - DNA, 19 bp, synthetic construct
  source 1..19: mol_type = other DNA
aacacaaagcatagactgc

SEQ ID NO: 22 - DNA, 20 bp, synthetic construct
  source 1..20: mol_type = other DNA
ggcccagactgagcacgtga

SEQ ID NO: 23 - DNA, 20 bp, synthetic construct
  source 1..20: mol_type = other DNA
gagtccgagcagaagaagaa

SEQ ID NO: 24 - DNA, 5028 bp, synthetic construct
  source 1..5028: mol_type = other DNA
atgggagtgcaggtggaaaccatctccccaggtgacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaccggctagctctgaggtggagttttcccacgagtactggatgagacatgccctgaccctggccaagagggcacgggatgagagggaggtgcctgtgggagccgtgctggtgctgaacaatagagtgatcggcgagggctggctgagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 25 - protein, 1676 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEPASSEVEFSHEYWMRHALTLAKRARDEREVPVGAVLVLNNRVIGEGWLRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

(19) United States
(12) Patent Application Publication
(10) Pub. No.: US 2024/0254508 A1
(43) Pub. Date: Aug. 1, 2024

(54) Multi-Genome Editor Construct and Uses Thereof

(71) Applicant: Colossal Biosciences Inc., Austin, TX (US)

(72) Inventors: Eriona Hysolli, Austin, TX (US); Rui Chen, Austin, TX (US); Daoyin Dong, Austin, TX (US); Michael Abrams, Austin, TX (US); Melissa Coquelin, Austin, TX (US)

(21) Appl. No.: 18/425,546

(22) Filed: Jan. 29, 2024

Related U.S. Application Data
(60) Provisional application No. 63/481,804, filed on Jan. 27, 2023.

(51) Int. Cl. C12N 15/85; C12N 9/22; C12N 15/11

(52) U.S. Cl. CPC C12N 15/85; C12N 9/22; C12N 15/11; C12N 2310/20; C12N 2800/90; C12N 2830/003

(57) ABSTRACT

Provided herein are isolated nucleic acids comprising two or more nucleotide sequences capable of encoding genome-editing enzymes. Also provided are vectors comprising the isolated nucleic acids, host cells comprising the vectors, and kits comprising the isolated nucleic acids.

### CROSS REFERENCE TO RELATED APPLICATIONS

This application claims priority to U.S. Provisional Application No. 63/481,804, filed Jan. 27, 2023, the disclosure of which is herein incorporated by reference in its entirety.

### FIELD OF THE INVENTION

The present disclosure generally relates to isolated nucleic acids capable of encoding at least two genome editing enzymes for use in increasing the editing modalities, and hence the multiplexability and efficiency of genome editing in a host genome.

### REFERENCE TO SEQUENCE LISTING SUBMITTED ELECTRONICALLY

This application contains a sequence listing, which is submitted electronically. The contents of the electronic sequence listing (069296.3US2 Sequence Listing.xml; Size: 117,472 bytes; and Date of Creation: Jan. 28, 2024) are herein incorporated by reference in its entirety.

### BACKGROUND OF THE INVENTION

Currently, many gene editing tools have been developed and utilized, such as CRISPR/Cas9 and its derivatives (base editor and prime editor, etc). Each gene editing tool has a specific application, field, and scope. For example, CRISPR/Cas9 can be used for imprecise gene disruption or precise gene editing by homology-directed repair (HDR). Base editors can be used for point mutations (C to T or A to G conversion) while prime editors can be used for nucleotide deletions, insertions, or substitutions.

In some cases, different gene editing tools are used for multiplexed gene editing in one cell. For example, an “A” gene may need to have a C to T conversion, a “B” gene may need an A to G conversion, a “C” gene may need a small deletion, a “D” gene may need to be disrupted, and/or an “E” gene may require a C to A conversion.

Additionally, the optimal choice for the specific type of gene editor needs to be decided for gene editing at a specific genomic locus. For example, base editors have a bystander effect at some genomic loci, and, thus, the base editors cannot be used to make point mutation in the genomic locus of interest. Instead, a prime editor may be a good choice, as a prime editor does not have a bystander effect. However, sometimes the best pegRNA and nicking gRNA for prime editing at a certain genomic locus cannot be determined. In this case, an HDR enzyme may be a better choice even though the HDR enzyme does not allow high throughput multiplexing editing.

Therefore, there is a need for one construct that is capable of performing each of these types of gene editing functions. A single construct in which expression of each of the genome editing enzymes can be controlled would represent an advance in the art.

### BRIEF SUMMARY OF THE INVENTION

Provided herein are isolated nucleic acids capable of encoding two or more genome editing enzymes. The isolated nucleic acids can, for example, comprise (a) a nucleotide sequence encoding a Cas enzyme; (b) a nucleotide sequence encoding a cytosine base editor (CBE); (c) a nucleotide sequence encoding an adenine base editor (ABE); (d) a nucleotide sequence encoding a cytosine to guanine base editor (CGBE); and/or (e) a nucleotide sequence encoding a neomycin resistance cassette; and a nucleotide sequence encoding regulatory elements for controlling expression of any one of nucleotide sequences (a), (b), (c), (d), or (c). In certain embodiments, the isolated nucleic acid further comprises (f) a nucleotide sequence encoding a guide ribonucleic acid (gRNA). In certain embodiments, the isolated nucleic acid comprises at least three of, at least four of, or all five of (a), (b), (c), (d), and/or (c). In certain embodiments, the isolated nucleic acid comprises at least three of, at least four of, at least five of, or all six of (a), (b), (c), (d), (c), and/or (f). In certain embodiments, the isolated nucleic acid further comprises (g) a nucleotide sequence encoding a prime editor (PE).

In certain embodiments, the isolated nucleic acids comprise (a) a nucleotide sequence encoding a Cas enzyme (e.g., CasX); (b) a nucleotide sequence encoding a cytosine base editor (CBE); (c) a nucleotide sequence encoding an adenine base editor (ABE); (d) a nucleotide sequence encoding a prime editor (PE); and/or (e) a nucleotide sequence encoding a neomycin resistance cassette; and a nucleotide sequence encoding regulatory elements for controlling expression of any one of nucleotide sequences (a), (b), (c), (d), or (c). In certain embodiments, the isolated nucleic acid further comprises (f) a nucleotide sequence encoding a guide ribonucleic acid (gRNA); or (g) a nucleotide sequence encoding a prime editor guide ribonucleic acid (pegRNA). In certain embodiments, the isolated nucleic acid comprises at least three of, at least four of, or all five of (a), (b), (c), (d), and/or (c). In certain embodiments, the isolated nucleic acid comprises at least three of, at least four of, at least five of, at least six of, or all seven of (a), (b), (c), (d), (c), (f), and/or (g). In certain embodiments, the isolated nucleic acid further comprises (h) a nucleotide sequence encoding a cytosine to guanine base editor (CGBE).

In certain embodiments, the Cas enzyme is Cas9. In certain embodiments, the nucleotide sequence encoding the Cas9 comprises a nucleotide sequence comprising at least 80% identity to the nucleotide sequence of SEQ ID NO:12. The nucleotide sequence encoding the Cas9 can, for example, encode an amino acid sequence comprising at least 90% identity to the amino acid sequence of SEQ ID NO:16. In certain embodiments, the nucleotide sequence encoding the Cas9 encodes the amino acid sequence of SEQ ID NO:16.

In certain embodiments, the nucleotide sequence encoding the CBE comprises a nucleotide sequence comprising at least 80% identity to the nucleotide sequence of SEQ ID NO:14. The nucleotide sequence encoding the CBE can, for example, encode an amino acid sequence comprising at least 90% identity to the amino acid sequence of SEQ ID NO:18. In certain embodiments, the nucleotide sequence encoding the CBE encodes the amino acid sequence of SEQ ID NO:18. In certain embodiments, the CBE further comprises a destabilization domain. The destabilization domain can, for example, be selected from a shield 1 destabilization domain or a trimethoprim (TMP) destabilization domain.

In certain embodiments, the nucleotide sequence encoding the ABE comprises a nucleotide sequence comprising at least 80% identity to the nucleotide sequence of SEQ ID NO:13. The nucleotide sequence encoding the ABE can, for example, encode an amino acid sequence comprising at least 90% identity to the amino acid sequence of SEQ ID NO:17. In certain embodiments, the nucleotide sequence encoding the ABE encodes the amino acid sequence of SEQ ID NO:17.

In certain embodiments, the nucleotide sequence encoding the CGBE comprises a nucleotide sequence with at least 80% identity to the nucleotide sequence of SEQ ID NO:15. The nucleotide sequence encoding the CGBE can, for example, encode an amino acid sequence comprising at least 90% identity to the amino acid sequence of SEQ ID NO:19. In certain embodiments, the nucleotide sequence encoding the CGBE encodes the amino acid sequence of SEQ ID NO:19. In certain embodiments, the CGBE further comprises a destabilization domain. The destabilization domain can, for example, be selected from a shield 1 destabilization domain or a trimethoprim (TMP) destabilization domain.

In certain embodiments, the Cas enzyme is CasX. In certain embodiments, the CasX further comprises a shield 1 destabilization domain. The nucleotide sequence encoding the CasX can, for example, comprise a nucleotide sequence comprising at least 80% identity to the nucleotide sequence of SEQ ID NO: 1. The nucleotide sequence encoding the CasX can, for example, encode an amino acid sequence comprising at least 90% identity to the amino acid sequence of SEQ ID NO:2. In certain embodiments, the nucleotide sequence encoding the CasX encodes the amino acid sequence of SEQ ID NO:2.

In certain embodiments, the nucleotide sequence encoding the CBE comprises a nucleotide sequence comprising at least 80% identity to the nucleotide sequence of SEQ ID NO:3. The nucleotide sequence encoding the CBE can, for example, encodes an amino acid sequence comprising at least 90% identity to the amino acid sequence of SEQ ID NO:4. In certain embodiments, the nucleotide sequence encoding the CBE encodes the amino acid sequence of SEQ ID NO:4. In certain embodiments, the CBE further comprises a destabilization domain. The destabilization domain can, for example, be selected from a shield 1 destabilization domain or a trimethoprim (TMP) destabilization domain.

In certain embodiments, the ABE further comprises a trimethoprim (TMP) destabilization domain. The nucleotide sequence encoding the ABE can, for example, comprise a nucleotide sequence comprising at least 80% identity to the nucleotide sequence of SEQ ID NO:5. The nucleotide sequence encoding the ABE can, for example, encode an amino acid sequence comprising at least 90% identity to the amino acid sequence of SEQ ID NO:6. In certain embodiments, the nucleotide sequence encoding the ABE encodes the amino acid sequence of SEQ ID NO:6.

In certain embodiments, the nucleotide sequence encoding the PE comprises a nucleotide sequence with at least 80% identity to the nucleotide sequence of SEQ ID NO:7. The nucleotide sequence encoding the PE can, for example, encode an amino acid sequence comprising at least 90% identity to the amino acid sequence of SEQ ID NO:8. In certain embodiments, the nucleotide sequence encoding the PE encodes the amino acid sequence of SEQ ID NO: 8. In certain embodiments, the PE further comprises a destabilization domain. The destabilization domain can, for example, be selected from a shield 1 destabilization domain or a trimethoprim (TMP) destabilization domain.

In certain embodiments, the nucleotide sequence encoding the neomycin resistance cassette comprises a nucleotide sequence comprising at least 80% identity to the nucleotide sequence of SEQ ID NO:9. The nucleotide sequence can, for example, encode the neomycin resistance cassette encodes an amino acid sequence comprising at least 90% identity to the amino acid sequence of SEQ ID NO:10. The nucleotide sequence can, for example, encode the neomycin resistance cassette encodes an amino acid sequence of SEQ ID NO:10.

In certain embodiments, the isolated nucleic acid comprises a nucleotide sequence having at least 80%, at least 85%, at least 90%, at least 95%, at least 96%, at least 97%, at least 98%, at least 99% identity to SEQ ID NO:11. In certain embodiments, the isolated nucleic acid comprises a nucleotide sequence of SEQ ID NO:11.

In certain embodiments, the Cas (e.g., Cas9 or CasX), ABE, CBE, and/or CGBE further comprise or are co-expressed with a non-interfering fluorescent protein. The non-interfering fluorescent protein can, for example, be selected from blue fluorescent protein (BFP), red fluorescent protein (RFP), green fluorescent protein (GFP), yellow fluorescent protein (YFP), or orange fluorescent protein. In certain embodiments, (1) the BFP is TagBFP; (2) the RFP is TagRFP657 or mCherry; and/or (3) the GFP is cGFP.

In certain embodiments, the Cas (e.g., Cas9 or CasX), ABE, CBE, and/or PE further comprise or are co-expressed with a non-interfering fluorescent protein. The non-interfering fluorescent protein is selected from mTagBFP2 (blue fluorescent protein), mRuby2 (basic red fluorescent protein), EGFP (enhanced green fluorescent protein), or mIFP (infrared fluorescent protein).

In certain embodiments, at least one of (a), (b), (c), (d), or (c) is operably linked to a promoter. The promoter can, for example, be a constitutive promoter or an inducible promoter. The constitutive promoter can, for example, be selected from an SV40 promoter, a CMV promoter, an EF-1A promoter, a UBC promoter, a PGK promoter, a CAG promoter, a CBh promoter, a CBA promoter, a U6 promoter, an H1 promoter, or a 7SK promoter. The inducible promoter can, for example, be selected from a chemically inducible promoter, a temperature inducible promoter, or a light inducible promoter. The chemically inducible promoter can, for example, be selected from tetracycline/doxycycline inducible promoter, a pLac inducible promoter, a pBad inducible promoter, a cumate inducible promoter, an alcohol inducible promoter, or a steroid inducible promoter. The temperature inducible promoter can, for example, be selected from an Hsp70 or Hsp90 promoter. The light inducible promoter can, for example, be selected from a UV light inducible promoter, a blue light inducible promoter, or a red/near-infrared (NIR) light inducible promoter.

In certain embodiments, the nucleotide sequence of (a), (b), (c), (d), or (c) further comprises a regulatory element capable of regulating the expression of the nucleotide sequence. The regulatory element can, for example, be selected from a cumate operator element or a tetracycline/doxycycline operator element.

In certain embodiments, for isolated nucleic acids encoding Cas (e.g., Cas9 or CasX), CBE, ABE, CGBE, and/or a neomycin resistance gene, (1) the nucleotide sequence of (a) is operably linked to a tetracycline/doxycycline inducible promoter; (2) the nucleotide sequence of (b) is operably linked to a CMV promoter; (3) the nucleotide sequence of (c) is operably linked to a cumate inducible promoter and further comprises a cumate operator element; (4) the nucleotide sequence of (d) is operably linked to the EF-1A promoter; or (5) the nucleotide sequence encoding the regulatory elements is operably linked to a EF-1A promoter.

In certain embodiments, for isolated nucleic acids encoding Cas (e.g., Cas 9 or CasX), CBE, ABE, PE, and/or a neomycin resistance gene, (1) the nucleotide sequence of (a) is operably linked to the PGK promoter; (2) the nucleotide sequence of (b) is operably linked to the CMV promoter and further comprising a cumate operator element; (3) the nucleotide sequence of (c) is operably linked to the CMV promoter; (4) the nucleotide sequence of (e) is operably linked to the EF-1A promoter; or (5) the nucleotide sequence encoding the regulatory elements is operably linked to the EF-1A promoter. In certain embodiments, the nucleotide sequence of (d) is operably linked to a tetracycline/doxycycline inducible promoter.

In certain embodiments, the regulatory elements comprise a rtTA transcription factor, a CymR repressor, and/or a tTA transcription factor.

Also provided are isolated nucleic acids comprising the following: (a) a nucleotide sequence encoding a Cas enzyme (e.g., Cas9), wherein the nucleotide sequence is operably linked to a tetracycline/doxycycline inducible promoter; (b) a nucleotide sequence encoding a cytosine base editor (CBE), wherein the nucleotide sequence is operably linked to a CMV promoter, and wherein the nucleotide sequence further comprises a trimethoprim (TMP) destabilization domain; (c) a nucleotide sequence encoding an adenine base editor (ABE), wherein the ABE further comprises a cumate operator element, and wherein the nucleotide sequence is operably linked to a cumate inducible promoter; (d) a nucleotide sequence encoding a cytosine to guanine base editor (CGBE), wherein the nucleotide sequence is operably linked to an EF-1A promoter, and wherein the CGBE further comprises a shield 1 destabilization domain; (c) a nucleotide sequence encoding a neomycin resistance gene, wherein the nucleotide sequence is operably linked to an EF-1A promoter; and a nucleotide sequence encoding regulatory elements for controlling expression of any one of nucleotide sequences (a), (b), (c), (d), or (c), and wherein the nucleotide sequence encoding the regulatory elements is operably linked to the same EF-1A promoter of (c). In certain embodiments, the isolated nucleic acid further comprises (f) a nucleotide sequence encoding a guide ribonucleic acid (gRNA), wherein the nucleotide sequence is operably linked to a U6 promoter.

Also provided are isolated nucleic acids comprising the following (a) a nucleotide sequence encoding a Cas enzyme (e.g., CasX), wherein the Cas enzyme further comprises a shield 1 destabilization domain, and wherein the nucleotide sequence is operably linked to a PGK promoter; (b) a nucleotide sequence encoding a cytosine base editor (CBE), wherein the nucleotide sequence is operably linked to a CMV promoter, and wherein the nucleotide sequence further comprises a cumate operator element; (c) a nucleotide sequence encoding an adenine base editor (ABE), wherein the ABE further comprises a trimethoprim (TMP) destabilization domain, and wherein the nucleotide sequence is operably linked to a CMV promoter; (d) a nucleotide sequence encoding a prime editor (PE), wherein the nucleotide sequence is operably linked to a tetracycline/doxycycline inducible promoter; (c) a nucleotide sequence encoding a neomycin resistance gene, wherein the nucleotide sequence is operably linked to an EF-1A promoter; and a nucleotide sequence encoding regulatory elements for controlling expression of any one of nucleotide sequences (a), (b), (c), (d), or (e), and wherein the nucleotide sequence is operably linked to the same EF-1A promoter of (c). In certain embodiments, the isolated nucleic acid further comprises (f) a nucleotide sequence encoding a guide ribonucleic acid (gRNA), wherein the nucleotide sequence is operably linked to a U6 promoter; or (g) a nucleotide sequence encoding a prime editor guide ribonucleic acid (pegRNA), wherein the nucleotide sequence is operably linked to a U6 promoter.

Also provided are isolated vectors comprising an isolated nucleic acid of the invention. The isolated vector can, for example, be selected from a PiggyBac (PB) plasmid, Sleep Beauty (SB) plasmid, or a Tol2 plasmid.

Also provided are hosts cells comprising an isolated vector of the invention.

Also provided are kits comprising (a) an isolated nucleic acid of the invention; (b) a regulatory molecule for controlling expression of any one of (a), (b), (c), (d), or (c) of the isolated nucleic acid; and (c) instructions for use. In certain embodiments, the regulatory molecule is selected from cumate, shield 1, trimethoprim (TMP), tetracycline, doxycycline, arabinose, isopropyl b-D-1-thiogalactopyranoside (IPTG), abscisic acid, gibberellin acid, and/or rapamycin.

### BRIEF DESCRIPTION OF THE DRAWINGS

The foregoing summary, as well as the following detailed description of preferred embodiments of the present disclosure, will be better understood when read in conjunction with the appended drawings. It should be understood, however, that the disclosure is not limited to the precise embodiments shown in the drawings.

FIGS. 1A and 1B show schematics of constructs comprising genome editing enzymes controlled by regulatory elements.

FIG. 1C shows a schematic of a construct comprising genome editing enzymes controlled by regulatory elements. The construct comprises four inducible modules as follows: (1) a TetOn promoter controlled, doxycycline induced spCas9, reported by a far red fluorescent protein (TagRFP657); (2) a cumate promoter controlled, cumate induced adenine base editor (ABE) (ABE8a spry), reported by a blue fluorescent protein (TagBFP); (3) an ecDHFR destabilization domain controlled, trimethroprim (TMP) induced cytosine base editor (CBE) (TadCBEd spry), conjugated and reported by a green fluorescent protein (eGFP); (4) an L106P mutant FKB12, destabilization domain controlled, Shield 1 induced cytosine to guanine based editor (CGBE) (tdCGBE spry), conjugated and reported by a red fluorescent protein (mCherry). There is an additional constitutively expressing cassette producing regulatory elements of the inducible modules (rtTA for TetOn promoter, CymR for cumate promoter) and a neomycin resistant gene as a selection marker. The mammalian expression sequence is flanked by piggyBac ITR sequences for genomic insertion.

FIG. 2 shows a schematic of the super editor map sequence as confirmed by nanopore sequencing.

FIGS. 3A-3D show fluorescence-activated cell sorting (FACS) of individual editor induction by super editor integrated HEK293T cells. FACS analysis was done for doxycycline induced Cas9 expression (FIG. 3A), cumate induced ABE expression (FIG. 3B), trimethoprim induced CBE expression (FIG. 3C), and shield 1 induced CGBE expression (FIG. 3D). Cells were fixed 72 hours after treatment for FACS quantification. Bar graphs plotting the induction ratios of each dose curve are provided for each different genome editor enzyme induction (FIGS. 3A-3D).

FIG. 4A shows Sanger sequencing chromatograms of HEK2 gene editing with recommended concentration of doxycycline induced Cas9 in super editor integrated HEK293T cells. Cells were treated with 1 μg/ml doxycycline. Cells were collected for PCR and Sanger sequencing 72 hours after inducer treatment and 48 hours after sgRNA plasmid transfection. The dashed line indicates the theoretical spCas9 cut site, and the bracket highlights the multi-peak sequences indicating spCas9 caused indels.

FIG. 4B shows Sanger sequencing chromatograms of HEK2 gene editing with recommended concentration of cumate induced ABE8e-spry in super editor integrated HEK293T cells. Cells were treated with 120 μM cumate. Cells were collected for PCR and Sanger sequencing 72 hours after inducer treatment and 48 hours after sgRNA plasmid transfection. The bottom chromatogram shows an example of editing resulting from transient constitutive ABE8e-spry expression. The arrows highlight the adenine to guanine substitution by the ABE8e-spry editor.

FIGS. 5A-5D show graphs demonstrating transient induction of single module transfection of genome editing enzymes in HEK293T cells. FIG. 5A shows a graph demonstrating doxycycline induced expression of Cas9 in HEK293T cells treated with 2 μg/ml of doxycycline. FIG. 5B shows a graph demonstrating cumate induced expression of ABE in HEK293T cells treated with 120 μg/ml of cumate. FIG. 5C shows a graph demonstrating trimethoprim (TMP) induced expression of CBE in HEK293T cells treated with 10 μM TMP. FIG. 5D shows a graph demonstrating shield 1 induced expression of CGBE in HEK293T cells treated with 6.25 μM shield 1.

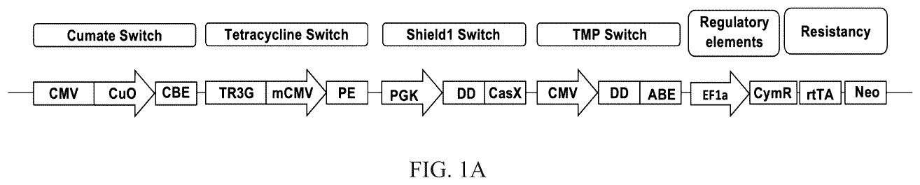

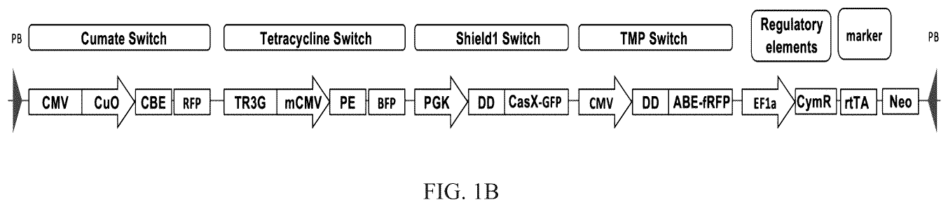

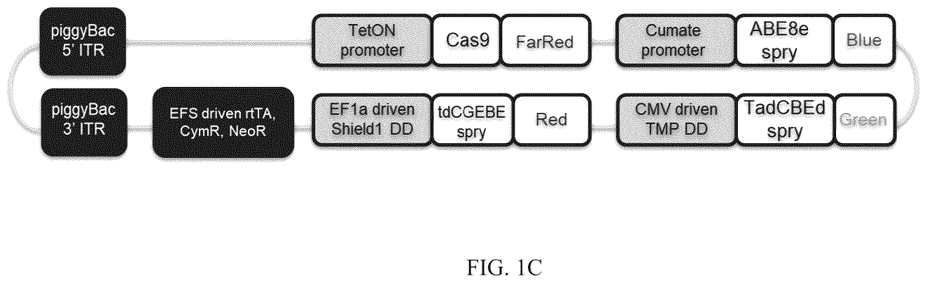

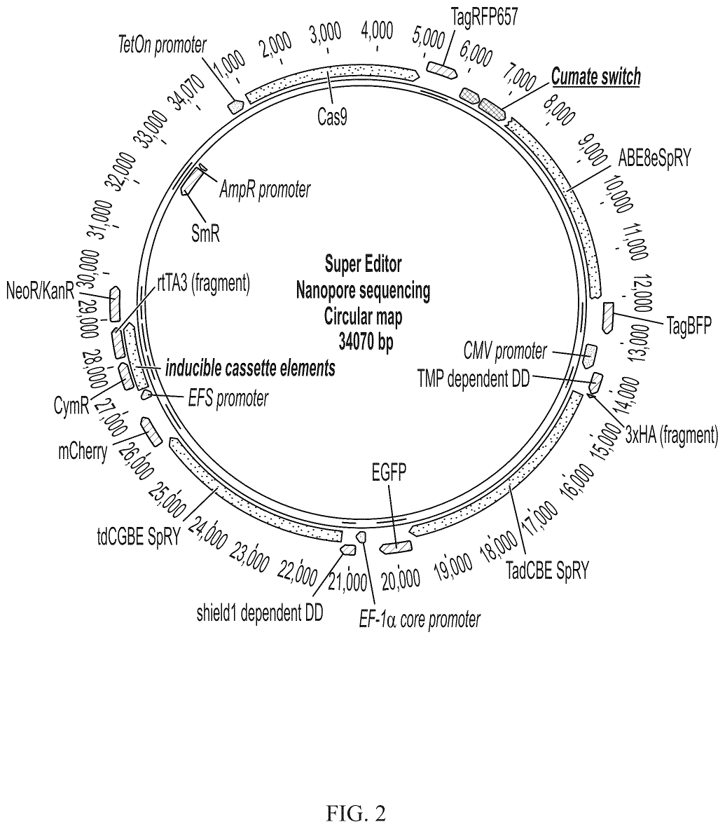

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., inv polypeptides and nucleotide sequences encoding the same, hly polypeptides and nucleotide sequences encoding the same), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the present disclosure, such as, for example an isolated vector comprising an isolated nucleic acid of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome. A host cell can be, for example, any type of prokaryotic, eukaryotic, or archaeal cell. In some instances, the host cell is a bacterial cell. In some instances, the host cell is a mammalian cell.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “heterologous nucleic acid” or “heterologous polypeptide” refers to a nucleic acid or a polypeptide whose sequence is not identical to that of another nucleic acid or polypeptide naturally found in the same host cell or the same host. As use herein, the “heterologous nucleic acid” or “heterologous polypeptide” can be heterologous to the bacterial cell and/or the mammalian host.

As used herein, the term “transform” or “transformation” refers to the transfer of a nucleic acid fragment into a host cell, such as a host bacterial cell, resulting in genetically-stable inheritance. Host cells comprising the transformed nucleic acid fragment are referred to as “recombinant” or “transgenic” or “transformed” organisms.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, “gene” refers to a nucleic acid comprising an open reading frame encoding a polypeptide, including both exon and (optionally) intron sequences.

As used herein, a “promoter” is an example of a transcriptional regulatory sequence and is specifically a nucleic acid sequence generally described as the proximal region of a gene located 5′ to the start codon. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

The term “expression cassette,” as used herein, refers to a nucleic acid construct comprising nucleic acid elements sufficient for the expression of a gene product, such as a polypeptide. Typically, an expression cassette comprises a nucleic acid encoding a gene product operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter. The term “heterologous promoter,” as used herein, refers to a promoter that is not found to be operatively linked to a given encoding sequence in nature. In some embodiments, an expression cassette may comprise additional elements, for example, an intron, an enhancer, a polyadenylation site, a woodchuck response element (WRE), and/or other elements known to affect expression levels of the encoding sequence. In some aspects, the expression cassette comprises at least one nucleotide sequence for insertion into a genome.

The term “gene product,” as used herein, refers to any product encoded by a nucleic acid sequence. Accordingly, a gene product may, for example, be a primary transcript, a mature transcript, a processed transcript, or a protein or peptide encoded by a transcript. Examples for gene products, accordingly, include mRNAs, rRNAs, hairpin RNAs (e.g. microRNAs, shRNAs, siRNAs, tRNAs), and peptides and proteins, for example, reporter proteins or therapeutic proteins.

### Genome Editing Constructs and Uses Thereof

Provided herein are inducible, reversible, tunable, and selectable genome editing constructs. The genome editing constructs can comprise genome editing enzymes, e.g., (a) a Cas enzyme for gene disruption or homology-directed repair (HDR); (b) a cytosine base editor (CBE) for cytosine (C) to thymidine (T) conversion; (c) an adenine base editor (ABE) for adenine (A) to guanosine (G) conversion; and/or (d) a prime editor (PE) for nucleotide deletion, insertion, and substitution. The genome editing constructs can also comprise genome editing enzymes, e.g., (a) a Cas enzyme for gene disruption or homology-directed repair (HDR); (b) a cytosine base editor (CBE) for cytosine (C) to thymidine (T) conversion; (c) an adenine base editor (ABE) for adenine (A) to guanosine (G) conversion; and/or (d) a cytosine to guanine base editor (CGBE) for cytosine (C) to guanine (G) conversion. An advantage of providing genome editing constructs expressing at least two genome editing enzymes, wherein the genome editing construct is capable of being inducible, reversible, tunable, and selectable, is that multiple genome edits can be made without the cross talk of multiple genome editing enzymes, as the precise expression of the precise enzyme can be controlled. Thus, different types of edits can be made, as a C to T conversion can be made when the CBE enzyme is induced to be expressed, and an A to G conversion can be made when the CBE enzyme is no longer being expressed and the ABE enzyme is induced to be expressed. This allows for multiple types of edits to occur without the cross talk of the genome editing enzymes.

The genome editing construct can, for example, be designed for controlling the expression of each genome editing enzyme (i.e., the Cas enzyme, the CBE, the ABE, the CGBE, and/or the PE) in an inducible, reversible, tunable, and selectable manner depending on which genome editing enzyme needs to be expressed, which is determined by the specific type of genome editing that is desired. By way of a non-limiting example, if a C to T conversion is desired, the CBE enzyme can be induced to express by providing a cell comprising the genome editing construct with a small molecule (e.g., cumate) that induces CBE expression, which in turn drives the C to T conversion in the genome at the specific target of interest. The expression of the CBE enzyme can be turned off by removing the small molecule (e.g., cumate) from the cell, which can allow for the selection of another genome editing enzyme for expression. Additionally, the expression of the CBE enzyme can be tuned (i.e., increased expression or decreased expression) by providing the cell with different amounts of the small molecule regulating expression of the enzyme (i.e., increased amounts would lead to increased expression of the CBE enzyme, and decreased amounts would lead to decreased expression of the CBE enzyme), thus allowing for the tunable expression of the CBE enzyme. By way of another non-limiting example, if an A to G conversion is desired, the ABE enzyme can be induced to express by providing a cell comprising the genome editing construct with a small molecule (e.g., cumate) that induces ABE expression, which in turn drives the A to G conversion in the genome at the specific target of interest. The expression of the ABE enzyme can be turned off by removing the small molecule (e.g., cumate) from the cell, which can allow for the selection of another genome editing enzyme for expression. Additionally, the expression of the ABE enzyme can be tuned (i.e., increased expression or decreased expression) by providing the cell with different amounts of the small molecule regulating expression of the enzyme (i.e., increased amounts would lead to increased expression of the ABE enzyme, and decreased amounts would lead to decreased expression of the ABE enzyme), thus allowing for the tunable expression of the ABE enzyme. By way of another non-limiting example, if a nucleotide deletion, insertion, or substitution is desired, the PE enzyme can be induced to express by providing the cell comprising the genome editing construct with a small molecule (e.g., tetracycline) that induces PE expression, which in turn drives the nucleotide deletion, insertion, or substitution in the genome at the specific target of interest. The expression of the PE enzyme can be turned off by removing the small molecule (e.g., tetracycline) from the cell, which can allow for the selection of another genome editing enzyme for expression. Additionally, the expression of the PE enzyme can be tuned (i.e., increased expression or decreased expression) by providing the cell with different amounts of the small molecule regulating expression of the enzyme (i.e., increased amounts would lead to increased expression of the PE enzyme, and decreased amounts would lead to decreased expression of the PE enzyme), thus allowing for the tunable expression of the PE enzyme. By way of another non-limiting example, if gene disruption or homology-directed repair (HDR) is desired, the Cas enzyme can be induced to express by providing the cell comprising the genome editing construct with a small molecule (e.g., tetracycline) that induces Cas expression, which in turn drives the gene disruption or homology-directed repair (HDR) in the genome at the specific target of interest. The expression of the Cas enzyme can be turned off by removing the small molecule (e.g., tetracycline) from the cell, which can allow for the selection of another genome editing enzyme for expression. Additionally, the expression of the Cas enzyme can be tuned (i.e., increased expression or decreased expression) by providing the cell with different amounts of the small molecule regulating expression of the enzyme (i.e., increased amounts would lead to increased expression of the Cas enzyme, and decreased amounts would lead to decreased expression of the Cas enzyme), thus allowing for the tunable expression of the Cas enzyme.

Thus, provided herein are isolated nucleic acids capable of encoding two or more genome editing enzymes. The isolated nucleic acids can, for example, comprise (a) a nucleotide sequence encoding a Cas enzyme; (b) a nucleotide sequence encoding a cytosine base editor (CBE); (c) a nucleotide sequence encoding an adenine base editor (ABE); (d) a nucleotide sequence encoding a cytosine to guanine base editor (CGBE); and/or (e) a nucleotide sequence encoding a neomycin resistance cassette; and a nucleotide sequence encoding regulatory elements for controlling expression of any one of nucleotide sequences (a), (b), (c), (d), or (c). In certain embodiments, the isolated nucleic acid further comprises (f) a nucleotide sequence encoding a guide ribonucleic acid (gRNA). In certain embodiments, the isolated nucleic acid comprises at least three of, at least four of, or all five of (a), (b), (c), (d), and/or (e). In certain embodiments, the isolated nucleic acid comprises at least three of, at least four of, at least five of, or all six of (a), (b), (c), (d), (c), and/or (f). In certain embodiments, the isolated nucleic acid further comprises (h) a nucleotide sequence encoding a prime editor (PE).

Also provided herein are isolated nucleic acids capable of encoding two or more genome editing enzymes. The isolated nucleic acids comprise (a) a nucleotide sequence encoding a Cas enzyme; (b) a nucleotide sequence encoding a cytosine base editor (CBE); (c) a nucleotide sequence encoding an adenine base editor (ABE); (d) a nucleotide sequence encoding a prime editor (PE); or (e) a nucleotide sequence encoding a neomycin resistance cassette; and a nucleotide sequence encoding regulatory elements for controlling expression of any one of nucleotide sequences (a), (b), (c), (d), or (c). In certain embodiments, the isolated nucleic acid further comprises (g) a nucleotide sequence encoding a guide ribonucleic acid (gRNA); or (f) a nucleotide sequence encoding a prime editor guide ribonucleic acid (pegRNA). In certain embodiments, the isolated nucleic acid comprises at least three of, at least four of, or all five of (a), (b), (c), (d), and/or (c). In certain embodiments, the isolated nucleic acid comprises at least three of, at least four of, at least five of, at least six of, or all seven of (a), (b), (c), (d), (c), (f), and/or (g). In certain embodiments, the isolated nucleic acid further comprises (h) a nucleotide sequence encoding a cytosine to guanine base editor (CGBE).

In certain embodiments, the isolated nucleic acid comprises a nucleotide sequence having at least 80%, at least 85%, at least 90%, at least 95%, at least 96%, at least 97%, at least 98%, at least 99% identity to SEQ ID NO:11. In certain embodiments, the isolated nucleic acid comprises a nucleotide sequence of SEQ ID NO:11.

As used herein, a “guide nucleic acid” or variants thereof can generally refer to a nucleic acid that may hybridize to another nucleic acid. A guide nucleic acid can be RNA. A guide nucleic acid can be DNA. The guide nucleic acid can be programmed to bind to a sequence of nucleic acid site-specifically, the sequence of nucleic acid can be referred to as the “target nucleic acid” or the “target.” A portion of the target nucleic acid can be complementary to a portion of the guide nucleic acid. The strand of a double started target nucleic acid that is complementary to and hybridizes with the guide nucleic acid may be called the complementary strand. The strand of the double-stranded target nucleic acid that is complementary with the complementary strand, and, therefore, not complementary with the guide nucleic acid, can be called the noncomplementary strand. A guide nucleic acid can comprise a segment that can be referred to as a “nucleic acid-targeting segment” or a “nucleic acid-targeting sequence.” A nucleic acid targeting segment can comprise a sub-segment that can be referred to as a “protein binding segment” or “protein binding sequence” or “Cas protein binding segment.”

A used herein, the term “guide RNA” can generally refer to an RNA molecule (or a group of RNA molecules collectively) that can bind to a Cas protein and aid in targeting the Cas protein a specific location within a target polynucleotide (e.g., a DNA). A guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5′-overhang sequence. As used herein the term “tracrRNA” or “tracrRNA segment” refers to an RNA molecule or portion thereof that includes a protein-binding segment (e.g., the protein-binding segment is capable of interacting with a CRISPR-associated protein, such as a Cas protein). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules.

As used herein, the term “CRISPR-associated protein” or “Cas protein” refers to a wild type Cas protein, a fragment thereof, or a mutant or variant thereof. The term “Cas mutant” or “Cas variant” refers to a protein or polypeptide derived from a wild type Cas protein, e.g., a protein having one or more point mutations, insertions, deletions, truncations, a fusion protein, or a combination thereof. In certain embodiments, the “Cas mutant” or “Cas variant” substantially retains the nuclease activity of the Cas protein. In certain embodiments, the “Cas mutant” or “Cas variant” is mutated in a way to render both nuclease domains inactive. In certain embodiments, the “Cas mutant” or “Cas variant” has nuclease activity. In certain embodiments, the “Cas mutant” or “Cas variant” lacks some or all of the nuclease activity of its wild-type counterpart.

### Cas Enzymes

The CRISPR/Cas system was first discovered in bacteria and archaea, where it functions as a form of adaptive immunity against viruses (Ishino et al., J. Bacteriol. 169(12):5429-33 (1997); Nakata et al., J. Bacteriol. 171(6):3553-6 (1989); Hermans et al., Infect. Immun. 59(8): 2695-2705 (1991); Mojica et al., Mol. Microbiol. 17:85-93 (1993); Jansen et al., Mol. Microbiol. 43(6): 1565-75 (2002); and Mojica et al., J. Mol. Evol. 60(2):174-82 (2005)). Cas proteins typically recognize small motifs (about 3-6 base pairs) present in the invading DNA, known as the protospacer-adjacent motif (PAM) (Gleditzsch et al., RNA Biol. 16(4):504-17 (2019)). The particular PAMs recognized differ among host species and are rarely present in the bacteria's own DNA to avoid self-cleavage. Following PAM recognition, a segment of downstream DNA (about 20 base pairs), known as a protospacer, is copied out of the foreign DNA and into a CRISPR array for transcription into short CRISPR RNAs (crRNAs) (Jinek et al., Science 337(6096):816-21 (2012)). These then anneal to trans-activating crRNAs (tracrRNAs) already present in the cell, which form a stem loop structure to allow Cas enzymes to bind. The RNA complex then acts as a guide for the Cas endonuclease to initiate sequence specific cleavage of foreign DNA, creating a DSB and silencing the pathogen (Jinek et al., Science 337(6096):816-21 (2012)). This system was adapted as a molecular tool with the crRNA/tracrRNA complex being replaced with a single guide RNA (sgRNA), which can be designed to target any desired sequence when introduced alongside a Cas protein to cleave at the target site.

Among different species of bacteria and archaea, a wide range of CRISPR/Cas systems have now been classified. The systems typically make use of different Cas endonucleases. Among the systems, Cas endonucleases show significant differences not only in their organization, but also in their size and functional structures. By using this diversity as a base for classification, it was recently suggested that CRISPR/Cas systems collectively form 2 classes, 6 types, and 33 subtypes (Makarova et al., Nat. Rev. Microbiol. 18(2):67-83 (2020)). Class I represents systems which generally contain multiple Cas enzymes collaboratively functioning to target DNA, and can be segregated into 3 types (I, III, and IV) and 16 subtypes (I-A, I-B, I-C, I-D, I-E, I-F, I-G, III-A, III-B, III-C, III-D, III-E, III-F, IV-A, IV-B, and IV-C). Due to the complexity of engineering and introducing multiple Cas enzymes into a cell, Class I systems are rarely used as genome editing tools.

In comparison, Class II systems typically require only a single, large, multifunctional Cas enzyme, making Class II systems simpler for adaptation. As a result, much research has been expended on developing current Class II systems and discovering more. Similar to Class I, in Class II, there are currently three (3) types (II, V, and IV) and 17 subtypes defined. Type II systems are the most well studied, following the early discovery of Cas9, which is currently the endonuclease most commonly used in CRISPR/Cas genome editing. CRISPR/Cas systems are described in a review by Li et al., J. Zhejiang University-Science B (Biomedicine & Biotechnology) 22(4):253-84 (2021), which is incorporated by reference herein.

Thus, provided herein are nucleic acids capable of encoding a Cas enzyme. In certain embodiments, the Cas enzyme is a Cas9 enzyme. In certain embodiments, the Cas9 further comprises a destabilization domain. The destabilization domain can, for example, be a shield 1 destabilization domain.

In certain embodiments, the nucleotide sequence encoding the Cas9 can, for example, comprise a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:12. In certain embodiments, the nucleotide sequence encoding Cas9 comprises the nucleotide of SEQ ID NO: 12.

In certain embodiments, the nucleotide sequence encoding the Cas9 can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 16. In certain embodiments, the nucleotide sequence encoding the Cas9 encodes the amino acid sequence of SEQ ID NO:16.

In certain embodiments, the Cas enzyme is a CasX enzyme. In certain embodiments, the CasX further comprises a destabilization domain. The destabilization domain can, for example, be a shield 1 destabilization domain.

The destabilization domain can confer instability to proteins-of-interest (e.g., the genome editing enzyme, i.e., Cas9, CasX, CBE, ABE, CGBE, and/or PE) when fused to the protein. When the destabilization domain is expressed in cells, the destabilization domain and the fusion protein are rapidly degraded by the proteasome, thus resulting in low levels of proteins-of-interest. Upon stabilization of the destabilization domain by addition of a small ligand, the degradation is reduced, which can induce a dose-dependent accumulation of the protein-of-interest within the cell. Thus, the level of the protein can be chemically controlled, creating a tool for the tunable expression of the protein-of-interest.

The compound shield 1 is a small molecule derived from a natural ligand FK506 and has been shown to interact selectively with the destabilization domain. Shield 1 is useful to protect the destabilization domain tagged proteins from proteasomal degradation, resulting in the rapid accumulation of the protein. Thus, the level of expression of the protein-of-interest can be controlled by the amount of shield 1 provided to a cell culture system expressing the protein-of-interest fused with the shield 1 destabilization domain, which allows for tuning the amount of stabilized protein-of-interest within the cells.

In certain embodiments, the nucleotide sequence encoding the CasX can, for example, comprise a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:1. In certain embodiments, the nucleotide sequence encoding CasX comprises the nucleotide sequence of SEQ ID NO:1.

In certain embodiments, the nucleotide sequence encoding the CasX can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:2. In certain embodiments, the nucleotide sequence encoding the CasX encodes the amino acid sequence of SEQ ID NO:2.

### Base Editors

Base editing is a breakthrough technology that can achieve single base transition or transversion precisely and efficiently at target sites without inducing double stranded breaks (DSBs) and the need for a donor repair template (DRT). Currently there are three base editors in use: cytosine base editors (CBEs) for C:G to T:A transition; adenine base editors (ABEs) for A:T to G:C transition; and C to G base editors (CGBEs) for C:G to G:C transversion. Precise base editing enables a single nucleotide substitution in a specific target gene to generate either a loss-of-function or gain-of-function mutation. Base editors are described in Li et al., J. Integrated Plant Biol. (2022).

### Cytosine Base Editor

The first-generation cytosine base editor (CBE) was engineered by fusing a rat cytidine deaminase (rAPOBEC1) to the N-terminus of an impaired dead Cas9 (dCas9) (Cas9 with D10A and H840A mutations) to generate rAPOBEC1-dCas9, which was designated as CBE1 (Komor et al., (2016)). The substitution of a C to a T in DNA is created by deaminating the cytosine (C) into a uracil (U) in the exposed non-target DNA strand, and the subsequent DNA repair and replication results in C to T base conversion. The cellular base excision repair (BER) mechanism enables C:G to T:A transition in vivo. The BER mechanism recognizes any G:U base pair as a mismatch, and the BER activity eliminates the uracil with the help of uracil N-glycosylase (UNG), resulting in a low efficiency of the CBE1 system (Komor et al., Nature 533:420-24 (2016)). To improve base editing efficiency, the second-generation base editor, CBE2 (rAPOBEC-dCas9-UGI), was constructed by binding a uracil DNA glycosylase inhibitor (UGI) to the C terminal of CBE1 to prevent the activity of UNG (Komor et al., Nature 533:420-24 (2016)). CBE2 improves editing efficiency and creates few unexpected indels. Subsequently, in order to further improve the editing efficiency, a third-generation cytosine base editor, CBE3, with an architecture of rAPOBEC1-nCas9(D10A)-UGI, was engineered by fusing the Cas9 nickase, nCas9(D10A), to rAPOBEC1 and UGI (Komor et al., Nature 533:420-24 (2016)). CBE3 cannot cut dsDNA, but CBE3 can create a nick in the target strand to incite the cellular repair process. Furthermore, in order to improve the deamination activity, a fourth-generation cytosine base editor, CBE4, was developed by fusing two UGI molecules to the C terminal of Cas9 nickase on the basis of CBE3 to enhance the inhibition of UNG (Komor et al., Sci. Adv. 3:caao4774 (2017)). Compared with CBE3, CBE4 not only improves the base editing efficiency but also reduces the frequency of C to A or G transversions by 2.3 times. In addition, bacteriophage Mu Gam protein was added on the basis of CBE4 to construct a base editor CBE4-Gam, in order to further improve the product purity and reduce the occurrence of indels (Komor et al., Sci. Adv. 3:caao4774 (2017)). The development of CBEs is described in Li et al., J. Integrated Plant Biol. (2022).

Thus, provided are nucleic acids capable of encoding a cytosine base editor (CBE) enzyme. In certain embodiments, the nucleotide sequence encoding the CBE comprises a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO: 14. In certain embodiments, the nucleotide sequence encoding the CBE comprises the nucleotide sequence of SEQ ID NO: 14.

In certain embodiments, the nucleotide sequence encoding the CBE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 18. In certain embodiments, the nucleotide sequence encoding the CBE encodes the amino acid sequence of SEQ ID NO:18.

In certain embodiments, the nucleotide sequence encoding the CBE comprises a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:3. In certain embodiments, the nucleotide sequence encoding the CBE comprises the nucleotide sequence of SEQ ID NO:3.

In certain embodiments, the nucleotide sequence encoding the CBE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:4. In certain embodiments, the nucleotide sequence encoding the CBE encodes the amino acid sequence of SEQ ID NO:4.

In certain embodiments, the CBE can, for example, further comprise a destabilization domain. The destabilization domain can, for example, be selected from a shield 1 destabilization domain or a trimethoprim (TMP) destabilization domain.

### Adenine Base Editor

Similar to CBE in both structure and base-editing mechanisms, adenine base editor (ABE) is composed of nCas9 (D10A) fused with an artificially evolved adenine deaminase, which helps to convert adenine (A) to inosine (I), and then DNA repair and replication to create A:T to G:C base substitution (Gaudelli et al., Nature 551:464-71 (2017)). ABE7.10 was engineered by fusing nCas9 (D10A) with a dimer composed of wild-type adenine deaminase TadA and an evolved adenine deaminase TadA7.10, with the editing window at positions of 4-8 nucleotides in the protospacer region (counting the PAM as positions 21-23 nucleotides). Subsequently, the editing efficiency of the ABE7.10 was increased by codon optimization and adding an additional nuclear localization sequence (NLS) in mammalian cell (Koblan et al., Nat. Biotechnol. 39:14114-25 (2018)). Furthermore, ABEmax was developed by adding an additional NLS at both ends of ABE7.10, with the editing efficiency of less than 50% at most target sites (Hua et al., Mol. Plant 11:627-30 (2018); Li et al., Genome Biol. 19:59 (2018); Yan et al., Mol. Plant 11:631-4 (2018)). A simplified base editor ABE-PIS containing TadA7.10-nCas9 (D10A) was also developed (Hua et al., Plant Biotechnol. J. 18:770-8 (2020)). ABE8e was further developed by using a more efficient adenine deaminase variant, TadA8e, which has been artificially evolved from TadA7.10 (Gaudelli et al., Nat. Biotechnol. 38:892-900 (2020); Richter et al., 2020). ABE8e deaminates the target base over a thousand times faster than the previous ABE7.10 and significantly improves the efficiency of A-to-G conversion (Richter et al., 2020). The mutation of V106W was also introduced in TadA8e to reduce the off-target effects (Richter et al., Nat. Biotechnol. 38:883-91 (2020)). A more efficient ABE toolbox (PhicABE) was developed based on hyTadA8e by fusing TadA8e and a single-stranded DNA-binding domain (DBD). The PhicABE has significantly higher base editing activity and broader editing windows compared with the general ABE8e systems (Tan et al., Plant Biotechnol. 20:934-43 (2022)). At last, a more efficient adenine deaminase, TadA9, was obtained in rice by incorporating two mutations, V82S and Q154R, into TadA8e (Yan et al., Mol. Plant. 14:722-31 (2021)). TadA9 is compatible with nSpCas9, nSpCas9-NG, and nScCas9, as well as near-PAMless SpRY. The development of ABEs is described in Li et al., J. Integrated Plant Biol. (2022).

Thus, provided herein are nucleic acids capable of encoding an adenine base editor (ABE). In certain embodiments, the ABE further comprises a destabilization domain. The destabilization domain can, for example, be a trimethoprim (TMP) destabilization domain, which is a mutant of the E. coli dihydrofolate reductase (ecDHER). The fusion of ecDHER to ABE enables the degradation of the fused protein. The addition of small molecule ligand TMP can bind to ecDHER and prevent ecDHER-ABE from degradation by proteasome. The interaction between TMP and ecDHER is specific and does not interfere with shield-1 destabilization domain.

In certain embodiments, the nucleotide sequence encoding the ABE comprises a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:13. In certain embodiments, the nucleotide sequence encoding the ABE comprises the nucleotide sequence of SEQ ID NO:13.

In certain embodiments, the nucleotide sequence encoding the ABE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In certain embodiments, the nucleotide sequence encoding the ABE encodes the amino acid sequence of SEQ ID NO: 17.

In certain embodiments, the nucleotide sequence encoding the ABE can, for example, comprise a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:5. In certain embodiments, the nucleotide sequence encoding the ABE comprises the nucleotide sequence of SEQ ID NO:5.

In certain embodiments, the nucleotide sequence encoding the ABE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:6. In certain embodiments, the nucleotide sequence encoding the ABE encodes the amino acid sequence of SEQ ID NO:6.

### Cytosine to Guanine Base Editor

Recently, new types of base editors, cytosine to guanine base editors (CGBEs) were created. CGBEs enable the cytosine transversions that are not capable of being made with cytosine base editors (CBEs) and adenosine base editors (ABEs). Examples of CGBEs are known in the art, see, e.g., Chen et al., Nat. Biotech. 41:663-72 (2023).

In certain embodiments, the nucleotide sequence encoding the CGBE comprises a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:15. In certain embodiments, the nucleotide sequence encoding the CGBE comprises the nucleotide sequence of SEQ ID NO:15.

In certain embodiments, the nucleotide sequence encoding the CGBE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:19. In certain embodiments, the nucleotide sequence encoding the ABE encodes the amino acid sequence of SEQ ID NO:19.

In certain embodiments, the CGBE further comprises a destabilization domain. The destabilization domain can, for example, be selected from a shield 1 destabilization domain or a trimethoprim (TMP) destabilization domain.

### Prime Editor

A search-and replace genome editing method, known as prime editing, has been developed to install precise small indels, all kinds of single or multiple base(s) substitutions (transitions and transversions) and their combinations at a target site in mammalian cells without requiring double stranded breaks (DSBs) and donor repair templates (DRTs). A prime editor is composed of a catalytically impaired nCas9 (H840A) fused with a reverse transcriptase, a M-MLV-RT (Moloney murine leukemia virus reverse transcriptase), at the C-terminus. A pegRNA composed of three components, including a sgRNA targeting the specific site, a reverse transcript encoding the desired edit as template (RTT), and a primer-binding site (PBS) initiating reverse transcription. In prime editing, the protein complex binds the target DNA and induces a nick at the non-target strand, from which the resulting 3′DNA terminal hybridizes to the PBS and then starts reverse transcription, to eventually copy the desired mutation into the genomic DNA following DNA replication and repair. Protein engineering and elaborated guide RNA designs contributed to the advent of several generations of PEs, from PE1 to PE2 and then to PE3 and PE3b, as well as the following generations with a gradual improvement in editing efficiency and/or product purity. PEs are described in Li et al., J. Integrated Plant Biol. (2022) and Chen et al., Cell 184:5635-52 (2021).

Thus, provided herein are nucleic acids capable of encoding a prime editor (PE) enzyme. In certain embodiments, the nucleotide sequence encoding the PE comprises a nucleotide sequence with at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:7. In certain embodiments, the nucleotide sequence encoding the PE comprises the nucleotide sequence of SEQ ID NO:7.

In certain embodiments, the nucleotide sequence encoding the PE can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:8. In certain embodiments, the nucleotide sequence encoding the PE encodes the amino acid sequence of SEQ ID NO: 8.

In certain embodiments, the PE can, for example, further comprise a destabilization domain. The destabilization domain can, for example, be selected from a shield 1 destabilization domain or a trimethoprim (TMP) destabilization domain.

### Selectable Markers

As used herein, “selectable marker” is an agent, such as a nucleic acid segment, that allows one to select for or against a molecule (e.g., a replicon) or a cell that contains it, often under particular conditions. These markers can encode an activity. Examples of selectable markers include but are not limited to: (1) nucleic acid sequences that encode products which provide resistance against otherwise toxic compounds (e.g., antibiotics); (2) nucleic acid sequences that encode products which are otherwise lacking in the recipient cell (e.g., tRNA genes, auxotrophic markers); (3) nucleic acid sequences that encode products which suppress the activity of a gene product; (4) nucleic acid sequences that encode products which can be readily identified (e.g., phenotypic markers such as β-galactosidase, green fluorescent protein (GFP), yellow fluorescent protein (YFP), cyan fluorescent protein (CFP), and cell surface proteins); (5) nucleic acid sequences that bind products which are otherwise detrimental to cell survival and/or function; (6) nucleic acid sequences that otherwise inhibit the activity of any of the nucleic acid sequences described in Nos. 1-5 above (e.g., antisense oligonucleotides); (7) nucleic acid sequences that bind products that modify a substrate (e.g. restriction endonucleases); (8) nucleic acid sequences that can be used to isolate or identify a desired molecule (e.g. specific protein binding sites); (9) nucleic acid sequences that encode a specific nucleotide sequence which can be otherwise non-functional (e.g., for PCR amplification of subpopulations of molecules); (10) nucleic acid sequences, which when absent, directly or indirectly confer resistance or sensitivity to particular compounds; and/or (11) nucleic acid sequences that encode products which are toxic in recipient cells. Examples of toxic gene products are well known in the art, and include, but are not limited to, restriction endonucleases (e.g., DpnI), apoptosis-related genes (e.g. ASK1 or members of the bcl-2/ced-9 family), retroviral genes including those of the human immunodeficiency virus (HIV), defensins such as NP-1, inverted repeats or paired palindromic nucleic acid sequences, bacteriophage lytic genes such as those from (ΦX174 or bacteriophage T4; antibiotic sensitivity genes such as rpsL, antimicrobial sensitivity genes such as pheS, plasmid killer genes, eukaryotic transcriptional vector genes that produce a gene product toxic to bacteria, such as GATA-1, and genes that kill hosts in the absence of a suppressing function, e.g., kicB, ccdB, ΦX174 E (Liu, Q. et al., Curr. Biol. 8:1300-1309 (1998), and other genes that negatively affect replicon stability and/or replication. A toxic gene can alternatively be selectable in vitro, e.g., a restriction site.

In certain embodiments, the isolated nucleic acids encode a selectable marker. The selectable marker can, for example, be a neomycin resistance cassette. In certain embodiments, the neomycin resistance cassette comprises a nucleotide sequence comprising at least 80%, at least 85%, at least 90%, at least 91%, at least 92%, at least 93%, at least 94%, at least 95%, at least 96%, at least 97%, at least 98%, or at least 99% identity to the nucleotide sequence of SEQ ID NO:9. In certain embodiments, the nucleotide sequence encoding the neomycin resistant cassette comprises the nucleotide sequence of SEQ ID NO:9.

In certain embodiments, the nucleotide sequence encoding the neomycin resistance cassette can, for example, encode an amino acid sequence comprising at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:10. The nucleotide sequence can, for example, encode the neomycin resistance cassette encodes an amino acid sequence of SEQ ID NO:10.

### Fluorescent Proteins

In certain embodiments, the Cas enzyme (e.g., Cas9 or CasX), ABE, CBE, and/or CGBE further comprise or are co-expressed with a non-interfering fluorescent protein. In certain embodiments, the nucleic acid capable of encoding at least two of the Cas enzyme (e.g., Cas9 or CasX), ABE, CBE, and/or CGBE can further encode a non-interfering fluorescent protein. In certain embodiments, the Cas enzyme (e.g., Cas9 or CasX), ABE, CBE, and/or PE further comprise or are co-expressed with a non-interfering fluorescent protein. In certain embodiments, the nucleic acid capable of encoding at least two of the Cas enzyme (e.g., Cas9 or CasX), ABE, CBE, and/or PE can further encode a non-interfering fluorescent protein. The non-interfering fluorescent protein can, for example, be selected from blue fluorescent protein (BFP), red fluorescent protein (RFP), green fluorescent protein (GFP), yellow fluorescent protein (YFP), or orange fluorescent protein. In certain embodiments, (1) the BFP is TagBFP; (2) the RFP is TagRFP657 or mCherry; and/or (3) the GFP is eGFP.

In certain embodiments, the Cas enzyme, ABE, CBE, and/or CGBE can be fused to a non-interfering fluorescent protein or can be co-expressed with a non-interfering fluorescent protein. The non-interfering fluorescent protein can, for example, be selected from blue fluorescent protein (BFP), red fluorescent protein (RFP), green fluorescent protein (GFP), yellow fluorescent protein (YFP), or orange fluorescent protein (OFP). The BFP can, for example, be TagBFP; the RFP can, for example, be TagRFP657 or mCherry; and the GFP can, for example, be cGFP. As used herein, “non-interfering” means that the fluorescent protein does not interfere with the enzyme function of the enzyme to which it is fused and/or co-expressed, and the fluorescent protein does not interfere with other fluorescent proteins in terms of the emission spectrums used for detection. Thus, the Cas enzyme, ABE, CBE, and/or CGBE fused to or co-expressed with the fluorescent protein can function at the same level as a wild-type Cas enzyme (e.g., Cas9 or CasX), ABE, CBE, and/or CGBE, and be detected by fluorescence-based cell sorting without interference with each other.

In certain embodiments, the Cas (e.g., Cas9 or CasX) and/or ABE enzyme can be fused to a non-interfering fluorescent protein, and the CBE and/or PE enzyme can be co-expressed with a non-interfering fluorescent protein. The non-interfering fluorescent protein is selected from mTagBFP2 (monomeric blue fluorescent protein with improved brightness and chemical stability), mRuby2 (monomeric variant of red fluorescent protein eqFP611), EGFP (enhanced green fluorescent protein), or mIFP (monomeric infrared fluorescent protein). As used herein, “non-interfering” means that the fluorescent protein does not interfere with the enzyme function of the enzyme to which it is fused and/or co-expressed, and the fluorescent protein does not interfere with other fluorescent proteins in terms of the emission spectrums used for detection. Thus, the Cas enzyme (e.g., Cas9 or CasX), ABE, CBE, and/or PE enzyme fused to or co-expressed with the fluorescent protein can function at the same level as a wild-type Cas enzyme (e.g., Cas9 or CasX), ABE, CBE, and/or PE, and be detected by fluorescence-based cell sorting without interference with each other.

In certain embodiments, the CasX is fused to or co-expressed with mTagBFP2, mRuby2, EGFP, and/or mIFP. In certain embodiments, the ABE is fused to or co-expressed with mTagBFP2, mRuby2, EGFP, and/or mIFP. In certain embodiments, the CBE is fused to or co-expressed with mTagBFP2, mRuby2, EGFP, and/or mIFP. In certain embodiments, the PE is fused to or co-expressed with mTagBFP2, mRuby2, EGFP, and/or mIFP. The inducible promoters are designed for editors which are co-expressed with a fluorescent protein. The destabilization domains are designed for editors which are fused with a fluorescent protein. By way of an example, the CasX can be fused to or co-expressed with mTagBFP2, the ABE can be fused to or co-expressed with mRuby2, the CBE can be fused to or co-expressed with EGFP, and the PE can be fused to or co-expressed with mIFP. By way of another example, the CasX can be fused to or co-expressed with EGFP, the ABE can be fused to or co-expressed with mTagBFP2, the CBE can be fused to or co-expressed with mRuby2, and the PE can be fused to or co-expressed with mIFP. By way of another example, the CasX can be fused to or co-expressed with EGFP, the ABE can be fused to or co-expressed with mIFP, the CBE can be fused to or co-expressed with mTagBFP2, and the PE can be fused to or co-expressed with mRuby2. By way of another example, the CasX can be fused to or co-expressed with EGFP, the ABE can be fused to or co-expressed with mRuby2, the CBE can be fused to or co-expressed with mIFP, and the PE can be fused to or co-expressed with mTagBFP2. A person skilled in the art can choose which fluorescent protein should be fused with which specific genome editor. For example, if a specific genome editing enzyme is not expressed at a high level, it can be fused to the brightest fluorescent protein (e.g., EGFP).

### Promoters and Regulatory Elements

In certain embodiments, at least one of (a), (b), (c), (d), or (e) is operably linked to a promoter. The promoter can, for example, be a constitutive promoter or an inducible promoter. The constitutive promoter can, for example, be selected from an SV40 promoter, a CMV promoter, an EF-1A promoter, a UBC promoter, a PGK promoter, a CAG promoter, a CBh promoter, a CBA promoter, a U6 promoter, an H1 promoter, or a 7SK promoter.

The inducible promoter can, for example, be selected from a chemically inducible promoter, a temperature inducible promoter, or a light inducible promoter. The chemically inducible promoter can, for example, be selected from tetracycline/doxycycline inducible promoter, a pLac inducible promoter, a pBad inducible promoter, a cumate inducible promoter, an alcohol inducible promoter, or a steroid inducible promoter. The temperature inducible promoter can, for example, be selected from an Hsp70 or Hsp90 promoter. The light inducible promoter can, for example, be selected from a UV light inducible promoter, a blue light inducible promoter, or a red/near-infrared (NIR) light inducible promoter.

In certain embodiments, the nucleotide sequence of (a), (b), (c), (d), or (e) further comprises a regulatory element capable of regulating the expression of the nucleotide sequence. The regulatory element can, for example, be selected from a cumate operator element or a tetracycline/doxycycline operator element.

Tetracycline selection technology allows for precise, reversible, and efficient spatiotemporal control of gene expression by binding directly to tTA or rtTA transcription factors. In a Tet-Off system, tetracycline (or its derivative doxycycline) prevents the tTA transcription factor from binding DNA at the promoter, thus inhibiting gene expression. In a Tet-On system, tetracycline binds to the rTA transcription factor and allows it to bind DNA at the promoter, thus inducing gene expression.

Thus, in certain aspects of the invention, the expression of a genome editing enzyme (e.g., Cas9, CasX, CBE, ABE, CGBE, and/or PE) can be controlled by a Tet-On system. The tetracycline response element combined with a mini-CMV promoter is placed upstream of the genome editing enzyme cassette (e.g., the Cas9, CasX, CBE, ABE, CGBE, and/or PE cassette). Treatment with tetracycline or doxycycline can induce the reversible expression of the genome editing enzyme in the cells.

The cumate switch system, which is an inducible gene expression system in mammalian cells, can control the expression of the genome editing enzyme of interest (e.g., Cas9, CasX, CBE, ABE, CGBE, and/or PE) through the addition of cumate to the system. Cumate is a non-toxic, small molecule inducer that can bind to CymR, which is a repressor that binds to cumate operator (CuO) sequences in the absence of cumate. With this system, expression levels can be tightly controlled, reversed, and increased with increasing cumate concentration until maximum induction is achieved. Importantly, background expression is negligible in the absence of cumate. The CMV5 promoter can, for example, hybridized with the cumate operator and placed upstream of the genome editing enzyme of interest (e.g., Cas9, CasX, CBE, ABE, CGBE, and/or PE). Treatment with cumate can induce the reversible expression of the genome editing enzyme of interest.

In certain embodiments, for isolated nucleic acids encoding (a) Cas enzyme (e.g., Cas9 or CasX), (b) CBE, (c) ABE, (d) CGBE, and (c) a neomycin resistance cassette, (1) the nucleotide sequence of (a) is operably linked to a tetracycline/doxycycline inducible promoter; (2) the nucleotide sequence of (b) is operably linked to a CMV promoter; (3) the nucleotide sequence of (c) is operably linked to a cumate inducible promoter and further comprises a cumate operator element; (4) the nucleotide sequence of (d) is operably linked to the EF-1A promoter; or (5) the nucleotide sequence encoding the regulatory elements is operably linked to a EF-1A promoter.

In certain embodiments, for isolated nucleic acids encoding (a) Cas enzyme (e.g., Cas or CasX), (b) CBE, (c) ABE, (d) PE, and (c) a neomycin resistance cassette, (1) the nucleotide sequence of (a) is operably linked to the PGK promoter; (2) the nucleotide sequence of (b) is operably linked to the CMV promoter and further comprising a cumate operator element; (3) the nucleotide sequence of (c) is operably linked to the CMV promoter; (4) the nucleotide sequence of (c) is operably linked to the EF-1A promoter; or (5) the nucleotide sequence encoding the regulatory elements is operably linked to the EF-1A promoter. In certain embodiments, the nucleotide sequence of (d) is operably linked to a tetracycline/doxycycline inducible promoter.

In certain embodiments, the regulatory elements comprise a rtTA transcription factor, a CymR repressor, and/or a tTA transcription factor.

Also provided are isolated nucleic acids comprising the following: (a) a nucleotide sequence encoding a Cas enzyme (e.g., Cas9), wherein the nucleotide sequence is operably linked to a tetracycline/doxycycline inducible promoter; (b) a nucleotide sequence encoding a cytosine base editor (CBE), wherein the nucleotide sequence is operably linked to a CMV promoter, and wherein the nucleotide sequence further comprises a trimethoprim (TMP) destabilization domain; (c) a nucleotide sequence encoding an adenine base editor (ABE), wherein the ABE further comprises a cumate operator element, and wherein the nucleotide sequence is operably linked to a cumate inducible promoter; (d) a nucleotide sequence encoding a cytosine to guanosine base editor (CGBE), wherein the nucleotide sequence is operably linked to an EF-1A promoter, and wherein the CGBE further comprises a shield 1 destabilization domain; (c) a nucleotide sequence encoding a neomycin resistance gene, wherein the nucleotide sequence is operably linked to an EF-1A promoter; and a nucleotide sequence encoding regulatory elements for controlling expression of any one of nucleotide sequences (a), (b), (c), (d), or (e), and wherein the nucleotide sequence encoding the regulatory elements is operably linked to the same EF-1A promoter of (c). In certain embodiments, the isolated nucleic acid further comprises (f) a nucleotide sequence encoding a guide ribonucleic acid (gRNA), wherein the nucleotide sequence is operably linked to a U6 promoter.

Also provided are isolated nucleic acids comprising the following (a) a nucleotide sequence encoding a Cas enzyme (e.g., CasX), wherein the Cas enzyme (e.g., CasX) further comprises a shield 1 destabilization domain, and wherein the nucleotide sequence is operably linked to a PGK promoter; (b) a nucleotide sequence encoding a cytosine base editor (CBE), wherein the nucleotide sequence is operably linked to a CMV promoter, and wherein the nucleotide sequence further comprises a cumate operator element; (c) a nucleotide sequence encoding an adenine base editor (ABE), wherein the ABE further comprises a trimethoprim (TMP) destabilization domain, and wherein the nucleotide sequence is operably linked to a CMV promoter; (d) a nucleotide sequence encoding a prime editor (PE), wherein the nucleotide sequence is operably linked to a tetracycline/doxycycline inducible promoter; (e) a nucleotide sequence encoding a neomycin resistance gene, wherein the nucleotide sequence is operably linked to an EF-1A promoter; and a nucleotide sequence encoding regulatory elements for controlling expression of any one of nucleotide sequences (a), (b), (c), (d), or (c), and wherein the nucleotide sequence is operably linked to the same EF-1A promoter of (e). In certain embodiments, the isolated nucleic acid further comprises (a) a nucleotide sequence encoding a guide ribonucleic acid (gRNA), wherein the nucleotide sequence is operably linked to a U6 promoter; or (b) a nucleotide sequence encoding a prime editor guide ribonucleic acid (pegRNA), wherein the nucleotide sequence is operably linked to a U6 promoter.

### Vectors and Host Cells

In another general aspect, the invention relates to a vector comprising the isolated nucleic acids of the invention. Any vector known to those skilled in the art in view of the present disclosure can be used, such as a plasmid, a cosmid, a phage vector, or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In certain aspects of the invention, the isolated vectors comprising the isolated nucleic acids of the invention can, for example, be selected from a PiggyBac (PB) plasmid, Sleep Beauty (SB) plasmid, or a Tol2 plasmid.

In another general aspect, the invention relates to a host cell comprising an isolated nucleic acid encoding two or more genome editing enzymes. Any host cell known to those skilled in the art in view of the present disclosure can be used for expression of the two or more genome editing enzymes. In some embodiments, the host cells are E. coli TG1 or BL21 cells, CHO-DG44 or CHO-K1 cells or HEK293 cells. According to particular embodiments, the recombinant expression vector is transformed into host cells by conventional methods such as chemical transfection, heat shock, or electroporation, where it can be stably integrated into the host cell genome such that the recombinant nucleic acid is effectively expressed.

In some aspects, the host cell is a mammalian cell to be edited. In some aspects, a cell useful in the methods and compositions described herein is an elephant cell. In some embodiments, the cell is an elephant fibroblast cell. In some embodiments, the cell is an elephant stem cell. In some aspects, the elephant cell is an African elephant cell. In some aspects, the elephant cell is an Asian elephant cell. In some embodiments, the cell described herein is an elephant somatic cell reprogrammed to a stem cell or stem cell-like phenotype having stem cell-like morphology and/or expressing at least one stem cell marker described herein. Examples of stem cell markers include, but are not limited to TRA 1-60, TRA 1-81, SSEA4, POU5F1, NANOG, REXI, hTERT, GDF3, miR-290 and mir-302 clusters among others for embryonic stem cells, and differentiation markers like SOX2, MYOD, PAX6, NESTIN, NEUROGENIN1/2, CD34, IL-7, IL-3, and NEUROD. In certain embodiments, the mammalian cell can, for example, be a hyrax cell or manatee cell. In another embodiment of any of the aspects, the hyrax cell is selected from the group consisting of: a Dendrohyrax arboreus cell, a Dendrohyrax dorsalis cell, a Heterohyrax brucei cell, and a Procavia capensis cell. In another embodiment, the manatee cell is selected from the group consisting of: a Trichechus inunguis cell, a Trichechus manatus cell, a Trichechus manatus latirostris cell, a Trichechus manatus manatus cell, and a Trichechus senegalensis cell. In some aspects, the cell is cryopreserved. In some aspects, the cell was previously cryopreserved.

In some aspects, the mammalian cells comprise stem cells. In some aspects, the mammalian cells comprise embryonic stem cells. In some aspects, the mammalian cells comprise Induced-pluripotent stem cells (iPSCs). In some aspects, the mammalian cells comprise Mesenchymal stem cells (MSCs). In some aspects, the mammalian cell comprises a fibroblast cell. In some aspects, the mammalian cell comprises a nerve cell, cartilage cell, bone cell, muscle cell, fat cell, or epidermal cell.

In some aspects, the mammalian cell line is a mammalian cell line obtainable from the American Type Culture Collection (Manassas, VA) and other depositories as well as commercial vendors. For instance, such mammalian cells include, but not limited to, MK2.7 cells, PER-C6 cells, Chinese hamster ovary cells (CHO), such as CHO-K1 (ATCC CCL-61), DG44 (Chasin et al., 1986, Som. Cell Molec. Genet., 12:555-556; Kolkekar et al., 1997, Biochemistry, 36:10901-10909; and WO 01/92337 A2), dihydrofolate reductase negative CHO cells (CHO/-DHFR. Urlaub and Chasin, 1980, Proc. Natl. Acad. Sci. USA, 77:4216), and dp 12.CHO cells (U.S. Pat. No. 5,721,121): monkey kidney cells (CV1, ATCC CCL-70); monkey kidney CV1 cells transformed by SV40 (COS cells, COS-7, ATCC CRL-1651); HEK 293 cells, and Sp2/0 cells, 5 L8 hybridoma cells, Daudi cells, EL4 cells, HeLa cells. HL-60 cells, K562 cells, Jurkat cells, THP-1 cells, Sp2/0 cells, primary epithelial cells (e.g., keratinocytes, cervical epithelial cells, bronchial epithelial cells, tracheal epithelial cells, kidney epithelial cells and retinal epithelial cells) and established cell lines and their strains (e.g., human embryonic kidney cells (e.g., 293 cells, or 293 cells subcloned for growth in suspension culture, Graham et al., 1977, J. Gen. Virol., 36:59); baby hamster kidney cells (BHK, ATCC CCL-10); mouse sertoli cells (TM4, Mather, 1980, Biol. Reprod., 23:243-251): human cervical carcinoma cells (HELA, ATCC CCL-2); canine kidney cells (MDCK, ATCC CCL-34): human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982, Annals NY Acad. Sci., 383:44-68); MCR 5 cells; FS4 cells; PER-C6 retinal cells, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells. T24 cells, WI-28 VA13, 2RA cells, WISH cells. BS—C-I cells, LLC-MK2 cells, Clone M-3 cells, 1-10 cells, RAG cells, TCMK-1 cells, Y-1 cells, LLC-PKI cells, PK(15) cells, GHI cells, GH3 cells, L2 cells, LLC-RC 256 cells, MHICI cells, XC cells, MDOK cells, VSW cells, and TH-I, BI cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCli cells, CV-1 cells, COS-1 cells, COS-3 cells, COS-7 cells, African green monkey kidney cells (VERO-76, ATCC CRL-1587; VERO, ATCC CCL-81); DBS-FrhL-2 cells, BALB/3T3 cells, F9 cells, SV-T2 cells, M-MSV-BALB/3T3 cells, K-BALB cells, BLO-11 cells, NOR-10 cells, C3H/IO/T2 cells, HSDMIC3 cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, CH cells, and Jensen cells, or derivatives thereof) or any other cell type known to one skilled in the art.

### Kits for Use

In one aspect of the invention, the disclosure generally relates to a kit comprising (a) an isolated nucleic acid of the invention; (b) a regulatory molecule for controlling expression of any one of (a), (b), (c), (d), or (c) of the isolated nucleic acid; and (c) instructions for use. In certain embodiments, the regulatory molecule is selected from cumate, shield 1, trimethoprim (TMP), tetracycline, doxycycline, arabinose, isopropyl b-D-1-thiogalactopyranoside (IPTG), abscisic acid, gibberellin acid, and/or rapamycin.

### EMBODIMENTS

The invention provides also the following non-limiting embodiments.

Embodiment 1 is an isolated nucleic acid comprising at least two of the following:
(a) a nucleotide sequence encoding a Cas enzyme;
(b) a nucleotide sequence encoding a cytosine base editor (CBE);
(c) a nucleotide sequence encoding an adenine base editor (ABE);
(d) a nucleotide sequence encoding a prime editor (PE); or
(e) a nucleotide sequence encoding a neomycin resistance cassette; and a nucleotide sequence encoding regulatory elements for controlling expression of any one of nucleotide sequences (a), (b), (c), (d), or (e).

Embodiment 2 is the isolated nucleic acid of embodiment 1, further comprising:
(f) a nucleotide sequence encoding a guide ribonucleic acid (gRNA); or
(g) a nucleotide sequence encoding a prime editor guide ribonucleic acid (pegRNA).

Embodiment 3 is the isolated nucleic acid of embodiment 1, wherein the nucleic acid comprises at least three of, at least four of, or all five of (a), (b), (c), (d), and/or (e).

Embodiment 4 is the isolated nucleic acid of embodiment 2, wherein the nucleic acid comprises at least three of, at least four of, at least five of, at least six of, or all seven of (a), (b), (c), (d), (e), (f), and/or (g).

Embodiment 4a is the isolated nucleic acid of any one of embodiments 1 to 4, further comprising (h) a nucleotide sequence encoding a cytosine to guanine base editor (CGBE).

Embodiment 4b is the isolated nucleic acid of any one of embodiments 1 to 4a, wherein the Cas enzyme is selected from Cas9 or CasX.

Embodiment 5 is the isolated nucleic acid of any one of embodiments 1 to 4b, wherein the CasX further comprises a shield 1 destabilization domain.

Embodiment 6 is the isolated nucleic acid of embodiment 5, wherein the nucleotide sequence encoding the CasX comprises a nucleotide sequence comprising at least 80% identity to the nucleotide sequence of SEQ ID NO:1.

Embodiment 7 is the isolated nucleic acid of embodiment 5 or 6, wherein the nucleotide sequence encoding the CasX encodes an amino acid sequence comprising at least 90% identity to the amino acid sequence of SEQ ID NO:2.

Embodiment 8 is the isolated nucleic acid of embodiment 7, wherein the nucleotide sequence encoding the CasX encodes the amino acid sequence of SEQ ID NO:2.

Embodiment 9 is the isolated nucleic acid of any one of embodiments 1 to 4, wherein the nucleotide sequence encoding the CBE comprises a nucleotide sequence comprising at least 80% identity to the nucleotide sequence of SEQ ID NO:3.

Embodiment 10 is the isolated nucleic acid of embodiment 9, wherein the nucleotide sequence encoding the CBE encodes an amino acid sequence comprising at least 90% identity to the amino acid sequence of SEQ ID NO:4.

Embodiment 11 is the isolated nucleic acid of embodiment 9 or 10, wherein the nucleotide sequence encoding the CBE encodes the amino acid sequence of SEQ ID NO:4.

Embodiment 12 is the isolated nucleic acid of any of embodiments 9 to 11, wherein the CBE further comprises a destabilization domain.

Embodiment 13 is the isolated nucleic acid of embodiment 12, wherein the destabilization domain is selected from a shield 1 destabilization domain or a trimethoprim (TMP) destabilization domain.

Embodiment 14 is the isolated nucleic acid of any one of embodiments 1 to 13, wherein the ABE further comprises a trimethoprim (TMP) destabilization domain.

Embodiment 15 is the isolated nucleic acid of embodiment 14, wherein the nucleotide sequence encoding the ABE comprises a nucleotide sequence comprising at least 80% identity to the nucleotide sequence of SEQ ID NO:5.

Embodiment 16 is the isolated nucleic acid of embodiment 14 or 15, wherein the nucleotide sequence encoding the ABE encodes an amino acid sequence comprising at least 90% identity to the amino acid sequence of SEQ ID NO:6.

Embodiment 17 is the isolated nucleic acid of embodiment 16, wherein the nucleotide sequence encoding the ABE encodes the amino acid sequence of SEQ ID NO:6.

Embodiment 18 is the isolated nucleic acid of any one of embodiments 1 to 17, wherein the nucleotide sequence encoding the PE comprises a nucleotide sequence with at least 80% identity to the nucleotide sequence of SEQ ID NO:7.

Embodiment 19 is the isolated nucleic acid of embodiment 18, wherein the nucleotide sequence encoding the PE encodes an amino acid sequence comprising at least 90% identity to the amino acid sequence of SEQ ID NO:8.

Embodiment 20 is the isolated nucleic acid of embodiment 18 or 19, wherein the nucleotide sequence encoding the PE encodes the amino acid sequence of SEQ ID NO: 8.

Embodiment 21 is the isolated nucleic acid of any one of embodiments 18 to 20, wherein the PE further comprises a destabilization domain.

Embodiment 22 is the isolated nucleic acid of embodiment 21, wherein the destabilization domain is selected from a shield 1 destabilization domain or a trimethoprim (TMP) destabilization domain.

Embodiment 23 is the isolated nucleic acid of any one of embodiments 1 to 22, wherein the nucleotide sequence encoding the neomycin resistance cassette comprises a nucleotide sequence comprising at least 80% identity to the nucleotide sequence of SEQ ID NO:9.

Embodiment 24 is the isolated nucleic acid of embodiment 23, wherein the nucleotide sequence encoding the neomycin resistance cassette encodes an amino acid sequence comprising at least 90% identity to the amino acid sequence of SEQ ID NO:10.

Embodiment 25 is the isolated nucleic acid of embodiment 23 or 24, wherein the nucleotide sequence encoding the neomycin resistance cassette encodes an amino acid sequence of SEQ ID NO:10.

Embodiment 26 is the isolated nucleic acid of any one of embodiments 1 to 25, wherein the CasX, ABE, CBE, and/or PE further comprise or are co-expressed with a non-interfering fluorescent protein.

Embodiment 27 is the isolated nucleic acid of embodiment 26, wherein the non-interfering fluorescent protein is selected from mTagBFP2, mRuby2, EGFP, or mIFP.

Embodiment 28 is the isolated nucleic acid of any one of embodiments 1-27, wherein at least one of (a), (b), (c), (d), or (e) is operably linked to a promoter.

Embodiment 29 is the isolated nucleic acid of embodiment 28, wherein the promoter is a constitutive promoter or an inducible promoter.

Embodiment 30 is the isolated nucleic acid of embodiment 29, wherein the constitutive promoter is selected from an SV40 promoter, a CMV promoter, an EF-1A promoter, a UBC promoter, a PGK promoter, a CAG promoter, a CBh promoter, a CBA promoter, a U6 promoter, an H1 promoter, or a 7SK promoter.

Embodiment 31 is the isolated nucleic acid of embodiment 29, wherein the inducible promoter is selected from a chemically inducible promoter, a temperature inducible promoter, or a light inducible promoter.

Embodiment 32 is the isolated nucleic acid of embodiment 31, wherein the chemically inducible promoter is selected from tetracycline/doxycycline inducible promoter, a pLac inducible promoter, a pBad inducible promoter, a cumate inducible promoter, an alcohol inducible promoter, or a steroid inducible promoter.

Embodiment 33 is the isolated nucleic acid of embodiment 31, wherein the temperature inducible promoter is selected from an Hsp70 or Hsp90 promoter.

Embodiment 34 is the isolated nucleic acid of embodiment 31, wherein the light inducible promoter is selected from a UV light inducible promoter, a blue light inducible promoter, or a red/near-infrared (NIR) light inducible promoter.

Embodiment 35 is the isolated nucleic acid sequence of embodiment 28, wherein the nucleotide sequence of (a), (b), (c), (d), or (e) further comprises a regulatory element capable of regulating the expression of the nucleotide sequence.

Embodiment 36 is the isolated nucleic acid sequence of embodiment 35, wherein the regulatory element is selected from a cumate operator element or a tetracycline/doxycycline operator element.

Embodiment 37 is the isolated nucleic acid of embodiment 30, wherein
(1) the nucleotide sequence of (a) is operably linked to the PGK promoter;
(2) the nucleotide sequence of (b) is operably linked to the CMV promoter and further comprising a cumate operator element;
(3) the nucleotide sequence of (c) is operably linked to the CMV promoter;
(4) the nucleotide sequence of (e) is operably linked to the EF-1A promoter; or
(5) the nucleotide sequence encoding the regulatory elements is operably linked to the EF-1A promoter.

Embodiment 38 is the isolated nucleic acid of embodiment 32, wherein the nucleotide sequence of (d) is operably linked to a tetracycline/doxycycline inducible promoter.

Embodiment 39 is the isolated nucleic acid of any one of embodiments 1-38, wherein the regulatory elements comprise a rtTA transcription factor, a CymR repressor, and/or a tTA transcription factor.

Embodiment 40 is an isolated nucleic acid comprising the following:
(a) a nucleotide sequence encoding a Cas enzyme (e.g., Cas 9 or CasX), wherein the Cas enzyme (e.g., Cas9 or CasX) further comprises a shield 1 destabilization domain, and wherein the nucleotide sequence is operably linked to a PGK promoter;
(b) a nucleotide sequence encoding a cytosine base editor (CBE), wherein the nucleotide sequence is operably linked to a CMV promoter, and wherein the nucleotide sequence further comprises a cumate operator element;
(c) a nucleotide sequence encoding an adenine base editor (ABE), wherein the ABE further comprises a trimethoprim (TMP) destabilization domain, and wherein the nucleotide sequence is operably linked to a CMV promoter;
(d) a nucleotide sequence encoding a prime editor (PE), wherein the nucleotide sequence is operably linked to a tetracycline/doxycycline inducible promoter;
(e) a nucleotide sequence encoding a neomycin resistance gene, wherein the nucleotide sequence is operably linked to an EF-1A promoter; and
a nucleotide sequence encoding regulatory elements for controlling expression of any one of nucleotide sequences (a), (b), (c), (d), or (e), and wherein the nucleotide sequence is operably linked to the same EF-1A promoter of (e).

Embodiment 41 is the isolated nucleic acid of embodiment 40, further comprising:
(f) a nucleotide sequence encoding a guide ribonucleic acid (gRNA), wherein the nucleotide sequence is operably linked to a U6 promoter; or
(g) a nucleotide sequence encoding a prime editor guide ribonucleic acid (pegRNA), wherein the nucleotide sequence is operably linked to a U6 promoter.

Embodiment 42 is an isolated vector comprising the isolated nucleic acid of any one of embodiments 1-41.

Embodiment 43 is the isolated vector of embodiment 42, wherein the vector is selected from a PiggyBac (PB) plasmid, Sleep Beauty (SB) plasmid, or a Tol2 plasmid.

Embodiment 44 is a host cell comprising the isolated vector of embodiment 42.

Embodiment 45 is a kit comprising:
(a) an isolated nucleic acid of any one of embodiments 1-41;
(b) a regulatory molecule for controlling expression of any one of (a), (b), (c), (d), or (e) of the isolated nucleic acid; and
(c) instructions for use.

Embodiment 46 is the kit of embodiment 45, wherein the regulatory molecule is selected from cumate, shield 1, trimethoprim (TMP), tetracycline, doxycycline, arabinose, isopropyl b-D-1-thiogalactopyranoside (IPTG), abscisic acid, gibberellin acid, and/or rapamycin.

Embodiment 47 is an isolated nucleic acid comprising at least two of the following:
(a) a nucleotide sequence encoding a Cas enzyme;
(b) a nucleotide sequence encoding a cytosine base editor (CBE);
(c) a nucleotide sequence encoding an adenine base editor (ABE);
(d) a nucleotide sequence encoding a cytosine to guanine base editor (CGBE); or
(e) a nucleotide sequence encoding a neomycin resistance cassette; and a nucleotide sequence encoding regulatory elements for controlling expression of any one of nucleotide sequences (a), (b), (c), (d), or (e).

Embodiment 48 is the isolated nucleic acid of embodiment 47, further comprising:
(f) a nucleotide sequence encoding a guide ribonucleic acid (gRNA).

Embodiment 49 is the isolated nucleic acid of embodiment 47, wherein the nucleic acid comprises at least three of, at least four of, or all five of (a), (b), (c), (d), and/or (e).

Embodiment 50 is the isolated nucleic acid of embodiment 48, wherein the nucleic acid comprises at least three of, at least four of, at least five of, or all six of (a), (b), (c), (d), (e), and/or (f).

Embodiment 50a is the isolated nucleic acid of any one of embodiments 47-50, further comprising (g) a nucleotide sequence encoding a prime editor (PE).

Embodiment 50b is the isolated nucleic acid of any one of embodiments 47-50a, wherein the Cas enzyme is selected from Cas9 or CasX.

Embodiment 51 is the isolated nucleic acid of claim any one of embodiments 47-50b, wherein the nucleotide sequence encoding the Cas9 comprises a nucleotide sequence comprising at least 80% identity to the nucleotide sequence of SEQ ID NO: 12.

Embodiment 52 is the isolated nucleic acid of any one of embodiments 47-51, wherein the nucleotide sequence encoding the Cas9 encodes an amino acid sequence comprising at least 90% identity to the amino acid sequence of SEQ ID NO:16.

Embodiment 53 is the isolated nucleic acid of embodiment 52, wherein the nucleotide sequence encoding the Cas9 encodes the amino acid sequence of SEQ ID NO:16.

Embodiment 54 is the isolated nucleic acid of any one of embodiments 47-53, wherein the nucleotide sequence encoding the CBE comprises a nucleotide sequence comprising at least 80% identity to the nucleotide sequence of SEQ ID NO: 14.

Embodiment 55 is the isolated nucleic acid of embodiment 54, wherein the nucleotide sequence encoding the CBE encodes an amino acid sequence comprising at least 90% identity to the amino acid sequence of SEQ ID NO:18.

Embodiment 56 is the isolated nucleic acid of embodiment 54 or 55, wherein the nucleotide sequence encoding the CBE encodes the amino acid sequence of SEQ ID NO:18.

Embodiment 57 is the isolated nucleic acid of any of embodiments 54 to 56, wherein the CBE further comprises a destabilization domain.

Embodiment 58 is the isolated nucleic acid of embodiment 57, wherein the destabilization domain is selected from a shield 1 destabilization domain or a trimethoprim (TMP) destabilization domain.

Embodiment 59 is the isolated nucleic acid of any one of claims 47-58, wherein the nucleotide sequence encoding the ABE comprises a nucleotide sequence comprising at least 80% identity to the nucleotide sequence of SEQ ID NO:13.

Embodiment 60 is the isolated nucleic acid of claim 59, wherein the nucleotide sequence encoding the ABE encodes an amino acid sequence comprising at least 90% identity to the amino acid sequence of SEQ ID NO:17.

Embodiments 61 is the isolated nucleic acid of embodiment 60, wherein the nucleotide sequence encoding the ABE encodes the amino acid sequence of SEQ ID NO:17.

Embodiment 62 is the isolated nucleic acid of any one of embodiments 47-61, wherein the nucleotide sequence encoding the CGBE comprises a nucleotide sequence with at least 80% identity to the nucleotide sequence of SEQ ID NO:15.

Embodiment 63 is the isolated nucleic acid of embodiment 62, wherein the nucleotide sequence encoding the CGBE encodes an amino acid sequence comprising at least 90% identity to the amino acid sequence of SEQ ID NO:19.

Embodiment 64 is the isolated nucleic acid of embodiment 62 or 63, wherein the nucleotide sequence encoding the CGBE encodes the amino acid sequence of SEQ ID NO:19.

Embodiments 65 is the isolated nucleic acid of any one of embodiments 62-64, wherein the CGBE further comprises a destabilization domain.

Embodiment 66 is the isolated nucleic acid of embodiment 65, wherein the destabilization domain is selected from a shield 1 destabilization domain or a trimethoprim (TMP) destabilization domain.

Embodiment 67 is the isolated nucleic acid of any one of embodiments 47-66, wherein the nucleotide sequence encoding the neomycin resistance cassette comprises a nucleotide sequence comprising at least 80% identity to the nucleotide sequence of SEQ ID NO:9.

Embodiment 68 is the isolated nucleic acid of embodiment 67, wherein the nucleotide sequence encoding the neomycin resistance cassette encodes an amino acid sequence comprising at least 90% identity to the amino acid sequence of SEQ ID NO:10.

Embodiment 69 is the isolated nucleic acid of embodiment 67 or 68, wherein the nucleotide sequence encoding the neomycin resistance cassette encodes an amino acid sequence of SEQ ID NO:10.

Embodiment 69a is the isolated nucleic acid of any one of embodiments 47 to 69, wherein the isolated nucleic acid comprises a nucleotide sequence having at least 80%, at least 85%, at least 90%, at least 95%, at least 96%, at least 97%, at least 98%, at least 99% identity to SEQ ID NO:11.

Embodiment 69b is the isolated nucleic acid of embodiment 69a, wherein the isolated nucleic acid comprises a nucleotide sequence of SEQ ID NO:11.

Embodiment 70 is the isolated nucleic acid of any one of claims 47-69, wherein the Cas9, ABE, CBE, and/or CGBE further comprise or are co-expressed with a non-interfering fluorescent protein.

Embodiment 71 is the isolated nucleic acid of embodiment 70, wherein the non-interfering fluorescent protein is selected from blue fluorescent protein (BFP), red fluorescent protein (RFP), green fluorescent protein (GFP), yellow fluorescent protein (YFP), or orange fluorescent protein.

Embodiment 72 is the isolated nucleic acid of embodiment 71, wherein:
(1) the BFP is TagBFP;
(2) the RFP is TagRFP657 or mCherry; and/or
(3) the GFP is eGFP.

Embodiment 73 is the isolated nucleic acid of any one of embodiments 47-72, wherein at least one of (a), (b), (c), (d), or (e) is operably linked to a promoter.

Embodiment 74 is the isolated nucleic acid of embodiment 73, wherein the promoter is a constitutive promoter or an inducible promoter.

Embodiment 75 is the isolated nucleic acid of embodiment 74, wherein the constitutive promoter is selected from an SV40 promoter, a CMV promoter, an EF-1A promoter, a UBC promoter, a PGK promoter, a CAG promoter, a CBh promoter, a CBA promoter, a U6 promoter, an Hl promoter, or a 7SK promoter.

Embodiment 76 is the isolated nucleic acid of embodiment 74, wherein the inducible promoter is selected from a chemically inducible promoter, a temperature inducible promoter, or a light inducible promoter.

Embodiment 77 is the isolated nucleic acid of embodiment 76, wherein the chemically inducible promoter is selected from tetracycline/doxycycline inducible promoter, a pLac inducible promoter, a pBad inducible promoter, a cumate inducible promoter, an alcohol inducible promoter, or a steroid inducible promoter.

Embodiment 78 is the isolated nucleic acid of embodiment 76, wherein the temperature inducible promoter is selected from an Hsp70 or Hsp90 promoter.

Embodiment 79 is the isolated nucleic acid of embodiment 76, wherein the light inducible promoter is selected from a UV light inducible promoter, a blue light inducible promoter, or a red/near-infrared (NIR) light inducible promoter.

Embodiment 80 is the isolated nucleic acid sequence of embodiment 73, wherein the nucleotide sequence of (a), (b), (c), (d), or (e) further comprises a regulatory element capable of regulating the expression of the nucleotide sequence.

Embodiment 81 is the isolated nucleic acid sequence of claim 80, wherein the regulatory element is selected from a cumate operator element or a tetracycline/doxycycline operator element.

Embodiment 82 is the isolated nucleic acid of embodiment 74, wherein
(1) the nucleotide sequence of (a) is operably linked to a tetracycline/doxycycline inducible promoter;
(2) the nucleotide sequence of (b) is operably linked to a CMV promoter;
(3) the nucleotide sequence of (c) is operably linked to a cumate inducible promoter and further comprises a cumate operator element;
(4) the nucleotide sequence of (d) is operably linked to the EF-1A promoter; or
(5) the nucleotide sequence encoding the regulatory elements is operably linked to a EF-1A promoter.

Embodiment 83 is the isolated nucleic acid of any one of embodiments 80-82, wherein the regulatory elements comprise a rtTA transcription factor, a CymR repressor, and/or a tTA transcription factor.

Embodiment 84 is an isolated nucleic acid comprising the following:
(a) a nucleotide sequence encoding a Cas enzyme (e.g., Cas9), wherein the nucleotide sequence is operably linked to a tetracycline/doxycycline inducible promoter;
(b) a nucleotide sequence encoding a cytosine base editor (CBE), wherein the nucleotide sequence is operably linked to a CMV promoter, and wherein the nucleotide sequence further comprises a trimethoprim (TMP) destabilization domain;
(c) a nucleotide sequence encoding an adenine base editor (ABE), wherein the nucleotide sequence is operably linked to a cumate inducible promoter;
(d) a nucleotide sequence encoding a cytosine to guanosine base editor (CGBE), wherein the nucleotide sequence is operably linked to an EF-1A promoter, and wherein the CGBE further comprises a shield 1 destabilization domain;
(e) a nucleotide sequence encoding a neomycin resistance gene, wherein the nucleotide sequence is operably linked to an EF-1A promoter; and a nucleotide sequence encoding regulatory elements for controlling expression of any one of nucleotide sequences (a), (b), (c), (d), or (e), and wherein the nucleotide sequence encoding the regulatory elements is operably linked to the same EF-1A promoter of (e).

Embodiment 85 is the isolated nucleic acid of embodiment 84, further comprising:

(f) a nucleotide sequence encoding a guide ribonucleic acid (gRNA), wherein the nucleotide sequence is operably linked to a U6 promoter.

Embodiment 86 is an isolated vector comprising the isolated nucleic acid of any one of embodiments 47-85.

Embodiment 87 is the isolated vector of embodiment 86, wherein the vector is selected from a PiggyBac (PB) plasmid, Sleep Beauty (SB) plasmid, or a Tol2 plasmid.

Embodiment 88 is a host cell comprising the isolated vector of embodiment 86 or 87. Embodiment 89 is a kit comprising:
(a) an isolated nucleic acid of any one of embodiments 47-85;
(b) a regulatory molecule for controlling expression of any one of (a), (b), (c), (d), or
(c) of the isolated nucleic acid; and
(c) instructions for use.

Embodiment 90 is the kit of embodiment 89, wherein the regulatory molecule is selected from cumate, shield 1, trimethoprim (TMP), tetracycline, doxycycline, arabinose, isopropyl b-D-1-thiogalactopyranoside (IPTG), abscisic acid, gibberellin acid, and/or rapamycin.

### EXAMPLES

### Example 1: Construction of Nucleic Acid Construct Capable of Encoding Genome Editing Enzymes

A nucleic acid construct capable of encoding genome editing enzymes is constructed by methods known in the art, e.g., the Gibson cloning method, Golden Gate cloning method, restriction enzyme based cloning method, or yeast assembly method. Nucleic acid fragments for the construct may be amplified from existing constructs by Polymerase Chain Reaction (PCR) using primers containing homology arms. Nucleic acid fragments for the construct may also be chemically synthesized and then further amplified by PCR using primers containing homology arms. Primers are designed by Geneious Prime software (Biomatters; Boston, MA) using the sequences of all fragments through its “Gibson/Homology Cloning” algorithm. The PCR-amplified fragments for the construct are assembled using NEBuilder HiFi DNA assembly protocol from New England Biolabs (New England Biolabs; Ipswich, MA). The assembly reaction is transformed into NEB Stable Competent E. coli cells. A single colony is picked and inoculated into LB medium for overnight culture, and then constructs are extracted from bacterial and purified by column-based methods. The correct construct is confirmed by colony PCR, restriction digestion, and whole plasmid sequencing.

### Example 2: Incorporation of Construct Capable of Encoding Genome Editing Enzymes in Cells

A PiggyBac integration method may be used for incorporation of a construct capable of encoding genome editing enzymes into the cellular genome. In this case, 200,000 cells are collected and transfected with 100 ng transposase mRNA and 400 ng of an isolated nucleic acid construct capable of encoding genome editing enzymes by chemical induced transfection (e.g., lipofectamine) or electroporation (e.g., Neon transfection system). An example of electroporation parameters is 1600 V/10 ms/3 pulses. After expanding the transfected cells, an optimized concentration of neomycin for selecting positive cells with incorporation of the construct is determined by comparing to cells transfected with transposase mRNA only. The optimized neomycin concentration is used for selecting cells with an integrated construct in the genome, and the cells that do not have integration of the construct will die due to the exposure to neomycin.

### Example 3: Demonstration of Genome Editing in Cells by Construct Capable of Encoding Genome Editing Enzymes

To induce the expression of editor of interest, the small molecule, which is designed for controlling the expression of each editor, is added into the culture medium one day before transfection (e.g., doxycycline for the primer editor (PE), cumate for the cytosine base editor (CBE), shield-1 for the CasX, and/or TMP for the adenine base editor (ABE)). On the day of transfection, a synthetic guide RNA, a plasmid expressing guide RNA, or nucleic acid constructs expressing guide RNA are transfected into the treated cells by chemical induced transfection (e.g., lipofectamine) or electroporation (e.g., Lonza Nucleofector technology (Lonza; Basel, Switzerland)). The small molecule is kept in the culture medium until the cells are collected. Cells expressing the editor will show the fluorescence signal under certain excitation light. Cells are collected or sampled two or more days after transfection. The genomic nucleic acid (genome DNA (gDNA)) is extracted from the collected cells. The gDNA region of interest for editing is amplified by PCR. The PCR product is analyzed by Sanger Sequencing and Next Generation Sequencing to determine the editing efficiency. To terminate the editing process in the cells, the small molecule is removed by adding new cell culture media to the cells.

### Example 4: Incorporation and Demonstration of Genome Editing in Cells by Construct Capable of Encoding Genome Editing Enzymes

Creation of Super editor construct: The construction of the Super Editor plasmid contained 2 steps. The first step was to generate 5 “entry clones” by Gibson assembly. The second step was to assemble the entry clones together by BsaI based Golden gate assembly to generate the Super Editor. The Super editor sequence was divided into 5 portions. Each of the 5 portions correlated with each entry clone. Each entry clone plasmid was constructed by Gibson assembly using 5 or 6 DNA fragments and the Neb Hifi Builder assembly kit. The cloning fragments were either PCR amplified or commercially synthesized. The entry clone plasmids were hosted by NEB10beta bacteria cells with ampicillin resistance. The 5 entry clones and a piggyBac plasmid backbone plasmid were compatible with BasI based Golden gate assembly. They were assembled together by the NEB Golden Gate assembly Mix under the commercial guidelines, hosted by NEB 10beta bacteria cells with spectinomycin resistance. By nanopore sequencing, it was demonstrated that the assembly product exhibited high purity and perfect sequence.

Incorporation of Super Editor in HEK293T cells: 300,000 HEK293T cells were transfected by 4 μg of Super Editor plasmid and 3 μg PiggyBac transposase plasmid using 10 μL of Lipofectamine 2000 according to the manufacturer's protocol in a 6-well plate format. 2 μg/mL doxycycline was supplemented to the cells 48 hours in advance. The transfected cells were enriched twice by Fluorescence-activated cell sorting (FACS) gated with doxycycline induced TagRFP657 (far red) fluorescent signals 1 week and 3 weeks after transfection. The 1st enrichment selected the top 0.2% (10,000 cells) of the TagRFP657+ population. The 2nd enrichment selected the top 5% (374,000 cells) of the TagRFP657+ population.

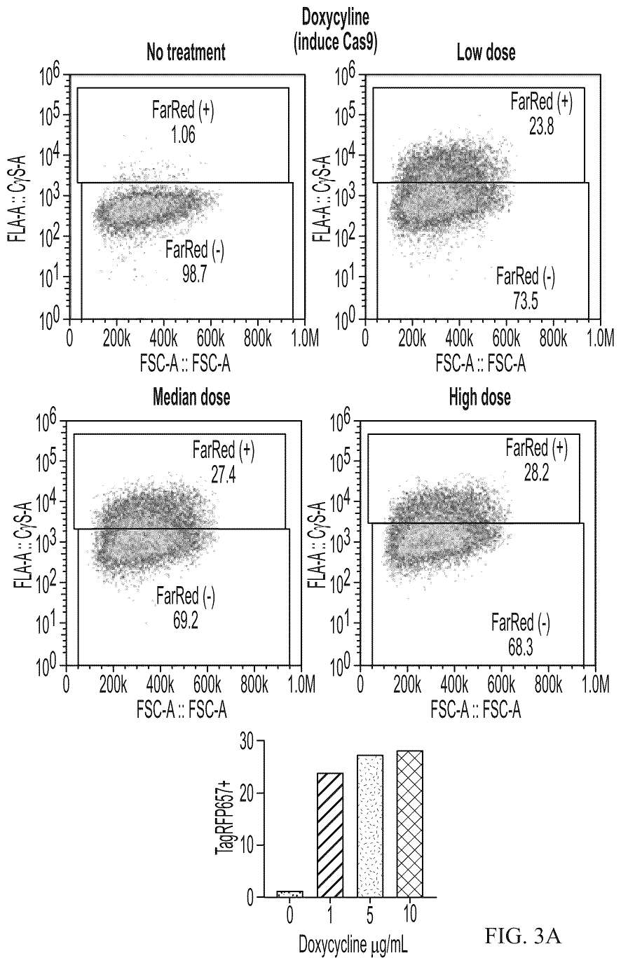

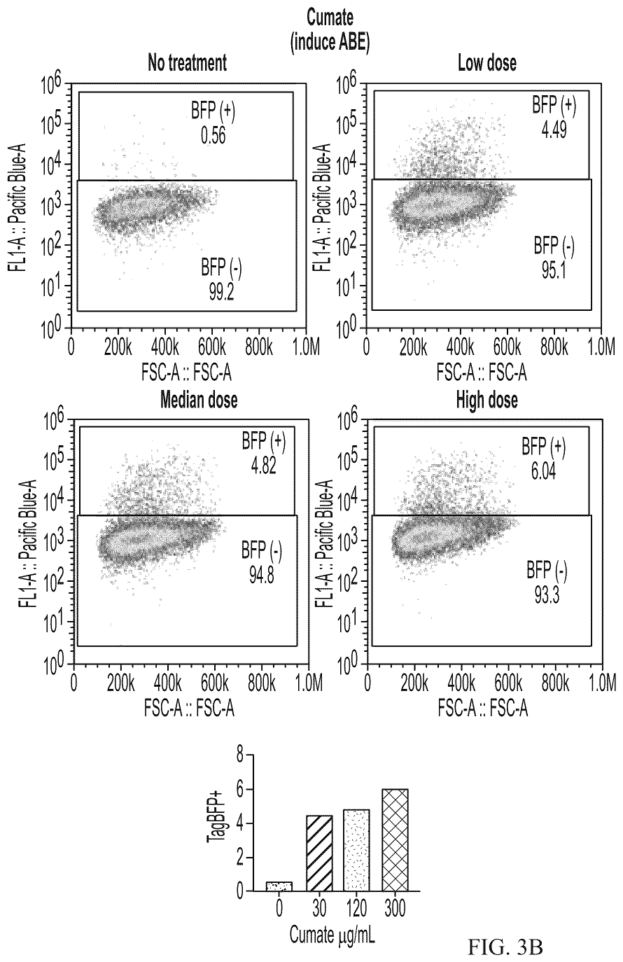

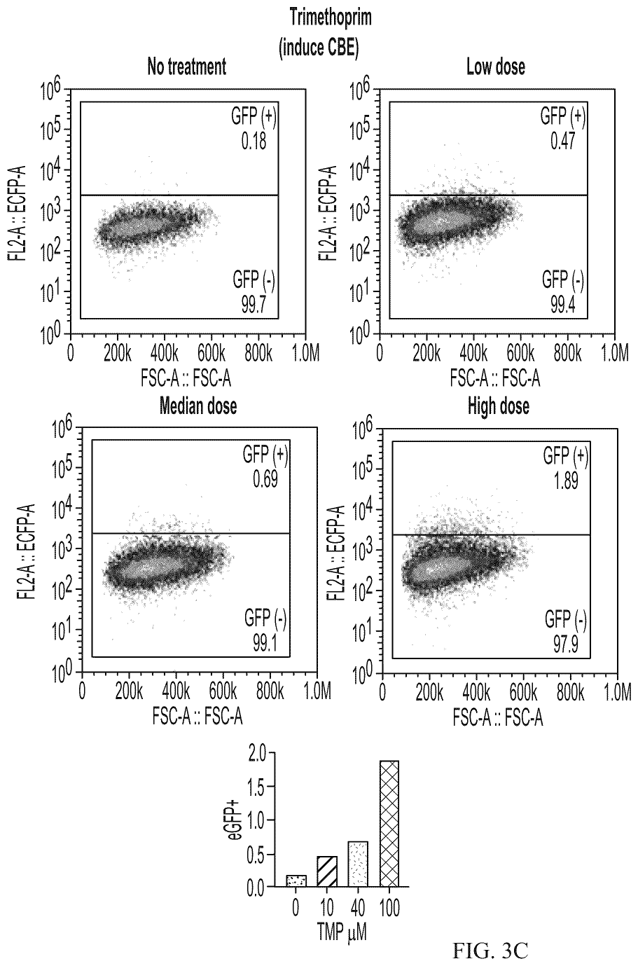

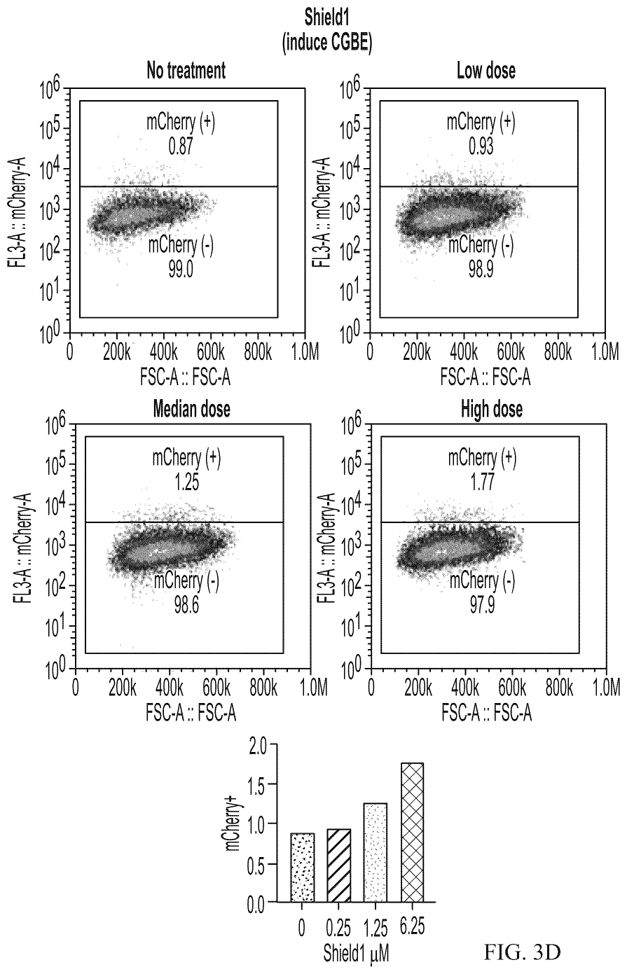

Demonstration of editing enzyme induction in Super Editor incorporated HEK293T cells: The following inducer concentration curves were tested separately in Super Editor incorporated HEK293T cells: 1/5/10 μg/mL of doxycycline, 30/120/300 μg/mL of cumate, 10/40/100 μM trimethoprim (TMP) and 0.25/1.25/6.25 μM Shield 1. Doxycycline induced Cas9, reported by TagRFP657 (far red); cumate induced ABE, reported by TagBFP (blue); trimethoprim induced CBE reported by, eGFP (green); Shield 1 induced CGBE, reported by mCherry (red). Cells were analyzed by fluorescent imaging or FACS 24-72 hours after treatment. FACS analysis was performed on 4% PFA fixed samples (FIGS. 3A-3D). Inducers were refreshed everyday with new media. As evaluated by induction levels/background signals/toxicity, recommended inducer concentrations were doxycycline 1 μg/mL, cumate 120 μg/mL, TMP 100 μM and Shield1 6.25 μM.

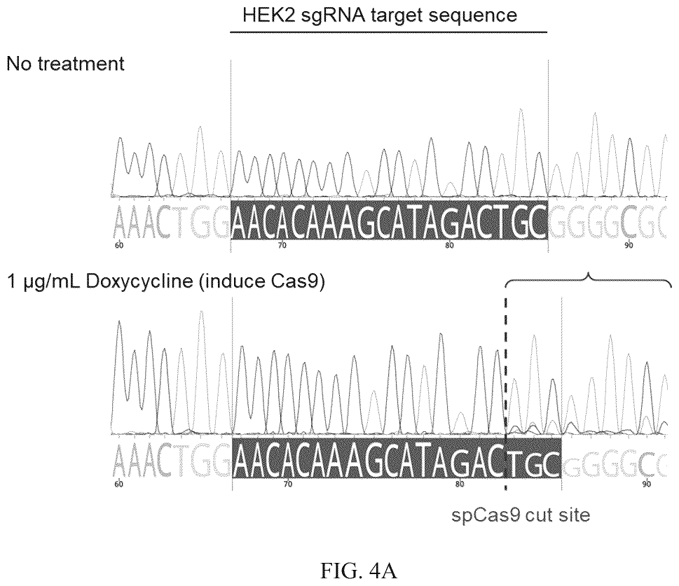

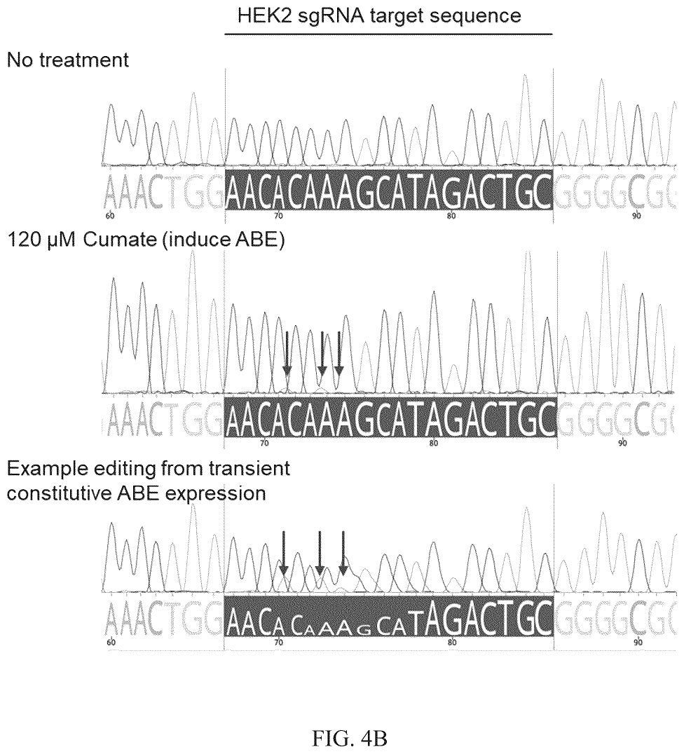

Demonstration of genome editing in Super Editor incorporated HEK293T cells: 200 k Super Editor incorporated HEK293T cells were supplemented with different inducers 24 hours before being transfected by 2 μg each sgRNA plasmid(s) using 4 μL of Lipofectamine 2000 according to the manufacturer's protocol in a 12-well plate format. Target loci included HBB (spacer sequence: GTAACGGCAGACTTCTCCTC (SEQ ID NO:20)), HEK2 (spacer sequence: AACACAAAGCATAGACTGC (SEQ ID NO:21)). Inducers were refreshed everyday with new media. Cells were collected 2 days after transfection for FACS analysis on 4% PFA fixed samples and gDNA extraction. The gDNA region with intended editing was amplified by PCR. The PCR product was analyzed by Sanger sequencing (FIGS. 4A and 4B).

Demonstration of transient editing enzyme induction and genome editing of Super editor in HEK293T cells: 1 million HEK293T cells were transfected by 1 μg Super Editor plasmid, 1 μg of sgRNA plasmid(s) using P3 Cell Line 4D-Nucleofector X Kit (Lonza; Basel, Switzerland) according to the manufacturer's protocol with large cuvette format. Target loci included HBB (spacer sequence: GTAACGGCAGACTTCTCCTC (SEQ ID NO:20)), HEK3 (spacer sequence: GGCCCAGACTGAGCACGTGA (SEQ ID NO:22)), EMX1 (spacer sequence: GAGTCCGAGCAGAAGAAGAA (SEQ ID NO:23)). 24 hours after transfection, cells were separately supplemented with inducers: doxycycline 1 μg/mL, or cumate 120 μg/mL, or TMP 100 μM, or Shield1 6.25 μM. Inducers were refreshed everyday with new media. Induction was monitored by fluorescent imaging. 3 days after transfection, live cells with positive corresponding fluorescent induction were analyzed by FACS and selectively sorted into gDNA extraction buffer. The gDNA region with intended editing was amplified by PCR. The PCR product was analyzed by Sanger sequencing.

Demonstration of transient editing enzyme induction and genome editing of single inducible module constructs in HEK293T cells: 200 k HEK293T cells were transfected by 2 μg single inducible module plasmid, 2 μg single inducible module regulator plasmid if necessary, and 1 μg of sgRNA plasmid(s) using 4 μL of Lipofectamine 2000 according to the manufacturer's protocol in a 12-well plate format. Target loci include HBB (spacer sequence: GTAACGGCAGACTTCTCCTC (SEQ ID NO:20)), HEK2 (spacer sequence: AACACAAAGCATAGACTGC (SEQ ID NO:21)), HEK3 (spacer sequence: GGCCCAGACTGAGCACGTGA (SEQ ID NO:22)), EMX1 (spacer sequence: GAGTCCGAGCAGAAGAAGAA (SEQ ID NO:23)). On the day of transfection, cells were separately supplemented with corresponding inducers: doxycycline 2 μg/mL, or cumate 120 μg/mL, or TMP 40 μM, or Shield1 6.25 μM. Inducers were refreshed everyday with new media. Induction was monitored by fluorescent imaging. Cells were collected 3 days after transfection for genomic gDNA extraction. The gDNA region with intended editing was amplified by PCR. The PCR product was analyzed by Sanger sequencing.

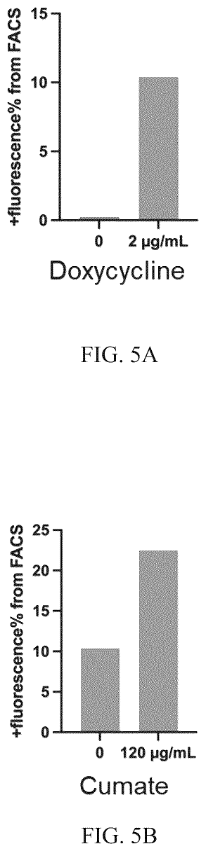

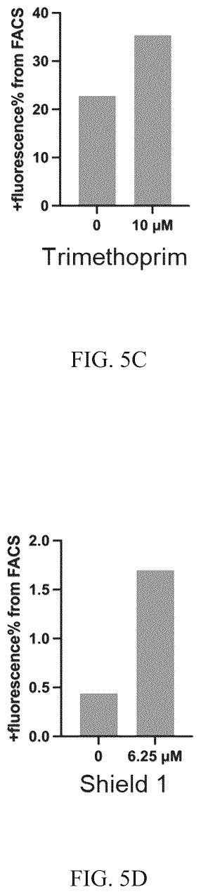

Demonstrating of transient induction of super editor corresponding to single module transfection in HEK293T cells: In order to check the quality of each inducible module, the inducible modules are clones as single module plasmids. HEK293T cells were transfected with the single module plasmids comprising the corresponding regulatory element. The HEK293T cells were treated with the corresponding inducers to generate expression of the genome editing enzyme along with the fluorescent marker accompanying each genome editing enzyme. HEK293T cells were transfected with a doxycycline inducible Cas9 editing enzyme, a cumate inducible ABE, a trimethoprim (TMP) inducible CBE, and a shield 1 inducible CGBE. After treatment with 2 μg/ml of doxycycline, 120 μg/ml of cumate, 10 μM TMP, and 6.25 μM shield 1, it was demonstrated that Cas9 and RFP were expressed (FIG. 5A), that ABE and BFP were expressed (FIG. 5B), that CBE and eGFP were expressed (FIG. 5C), and CGBE and mCherry were expressed (FIG. 5D).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated nucleic acid comprising at least two of the following:
(a) a nucleotide sequence encoding a Cas enzyme;
(b) a nucleotide sequence encoding a cytosine base editor (CBE);
(c) a nucleotide sequence encoding an adenine base editor (ABE);
(d) a nucleotide sequence encoding a cytosine to guanine base editor (CGBE); or
(e) a nucleotide sequence encoding a neomycin resistance cassette; and
a nucleotide sequence encoding regulatory elements for controlling expression of any one of nucleotide sequences (a), (b), (c), (d), or (e).

2. The isolated nucleic acid of claim 1, further comprising:
(f) a nucleotide sequence encoding a guide ribonucleic acid (gRNA).

3. (canceled)

4. The isolated nucleic acid of claim 2, wherein the nucleic acid comprises at least three of, at least four of, at least five of, or all six of (a), (b), (c), (d), (e), and/or (f).

5. The isolated nucleic acid of claim 1, wherein the Cas enzyme is a Cas9 or CasX enzyme.

6. The isolated nucleic acid of claim 5, wherein:
(a) the nucleotide sequence encoding the Cas9 comprises a nucleotide sequence comprising at least 80% identity to the nucleotide sequence of SEQ ID NO: 12;
(b) the nucleotide sequence encoding the CBE comprises a nucleotide sequence comprising at least 80% identity to the nucleotide sequence of SEQ ID NO:14;
(c) the nucleotide sequence encoding the ABE comprises a nucleotide sequence comprising at least 80% identity to the nucleotide sequence of SEQ ID NO:13;
(d) the nucleotide sequence encoding the CGBE comprises a nucleotide sequence with at least 80% identity to the nucleotide sequence of SEQ ID NO:15; or
(e) the nucleotide sequence encoding the neomycin resistance cassette comprises a nucleotide sequence comprising at least 80% identity to the nucleotide sequence of SEQ ID NO:9.

7. The isolated nucleic acid of claim 5, wherein the nucleotide sequence encoding the Cas9 encodes an amino acid sequence comprising at least 90% identity to the amino acid sequence of SEQ ID NO:16.

8-9. (canceled)

10. The isolated nucleic acid of claim 6, wherein the nucleotide sequence encoding the CBE encodes an amino acid sequence comprising at least 90% identity to the amino acid sequence of SEQ ID NO:18.

11. (canceled)

12. The isolated nucleic acid of claim 6, wherein the CBE further comprises a destabilization domain, wherein the destabilization domain is selected from a shield 1 destabilization domain or a trimethoprim (TMP) destabilization domain.

13-14. (canceled)

15. The isolated nucleic acid of claim 6, wherein the nucleotide sequence encoding the ABE encodes an amino acid sequence comprising at least 90% identity to the amino acid sequence of SEQ ID NO:17.

16-17. (canceled)

18. The isolated nucleic acid of claim 6, wherein the nucleotide sequence encoding the CGBE encodes an amino acid sequence comprising at least 90% identity to the amino acid sequence of SEQ ID NO:19.

19. (canceled)

20. The isolated nucleic acid of claim 6, wherein the CGBE further comprises a destabilization domain, wherein the destabilization domain is selected from a shield 1 destabilization domain or a trimethoprim (TMP) destabilization domain.

21-22. (canceled)

23. The isolated nucleic acid of claim 6, wherein the nucleotide sequence encoding the neomycin resistance cassette encodes an amino acid sequence comprising at least 90% identity to the amino acid sequence of SEQ ID NO:10.

24. (canceled)

25. The isolated nucleic acid of claim 1, wherein the Cas, ABE, CBE, and/or CGBE further comprise or are co-expressed with a non-interfering fluorescent protein.

26. The isolated nucleic acid of claim 25, wherein the non-interfering fluorescent protein is selected from blue fluorescent protein (BFP), red fluorescent protein (RFP), green fluorescent protein (GFP), yellow fluorescent protein (YFP), or orange fluorescent protein.

27. (canceled)

28. The isolated nucleic acid of claim 1, wherein at least one of (a), (b), (c), (d), or (e) is operably linked to a promoter.

29. (canceled)

30. The isolated nucleic acid of claim 28, wherein the promoter is a constitutive promoter, and wherein the constitutive promoter is selected from an SV40 promoter, a CMV promoter, an EF-1A promoter, a UBC promoter, a PGK promoter, a CAG promoter, a CBh promoter, a CBA promoter, a U6 promoter, an H1 promoter, or a 7SK promoter.

31. The isolated nucleic acid of claim 28, wherein the promoter is an inducible promoter, and wherein the inducible promoter is selected from a chemically inducible promoter, a temperature inducible promoter, or a light inducible promoter.

32. The isolated nucleic acid of claim 31, wherein the chemically inducible promoter is selected from tetracycline/doxycycline inducible promoter, a pLac inducible promoter, a pBad inducible promoter, a cumate inducible promoter, an alcohol inducible promoter, or a steroid inducible promoter.

33-34. (canceled)

35. The isolated nucleic acid sequence of claim 28, wherein the nucleotide sequence of (a), (b), (c), (d), or (e) further comprises a regulatory element capable of regulating the expression of the nucleotide sequence.

36. The isolated nucleic acid sequence of claim 35, wherein the regulatory element is selected from a cumate operator element or a tetracycline/doxycycline operator element.

37. The isolated nucleic acid of claim 29, wherein
(1) the nucleotide sequence of (a) is operably linked to a tetracycline/doxycycline inducible promoter;
(2) the nucleotide sequence of (b) is operably linked to a CMV promoter;
(3) the nucleotide sequence of (c) is operably linked to a cumate inducible promoter;
(4) the nucleotide sequence of (d) is operably linked to the EF-1A promoter; or
(5) the nucleotide sequence encoding the regulatory elements is operably linked to a EF-1A promoter.

38. The isolated nucleic acid of claim 35, wherein the regulatory elements comprise a rtTA transcription factor, a CymR repressor, and/or a tTA transcription factor.

39. An isolated nucleic acid comprising the following:
(a) a nucleotide sequence encoding a Cas enzyme, wherein the nucleotide sequence is operably linked to a tetracycline/doxycycline inducible promoter;
(b) a nucleotide sequence encoding a cytosine base editor (CBE), wherein the nucleotide sequence is operably linked to a CMV promoter, and wherein the nucleotide sequence further comprises a trimethoprim (TMP) destabilization domain;
(c) a nucleotide sequence encoding an adenine base editor (ABE), wherein the nucleotide sequence is operably linked to a cumate inducible promoter;
(d) a nucleotide sequence encoding a cytosine to guanine base editor (CGBE), wherein the nucleotide sequence is operably linked to an EF-1A promoter, and wherein the CGBE further comprises a shield 1 destabilization domain;
(e) a nucleotide sequence encoding a neomycin resistance gene, wherein the nucleotide sequence is operably linked to an EF-1A promoter; and
a nucleotide sequence encoding regulatory elements for controlling expression of any one of nucleotide sequences (a), (b), (c), (d), or (e), and wherein the nucleotide sequence encoding the regulatory elements is operably linked to the same EF-1A promoter of (e).

40. The isolated nucleic acid of claim 39, further comprising:
(f) a nucleotide sequence encoding a guide ribonucleic acid (gRNA), wherein the nucleotide sequence is operably linked to a U6 promoter.

41. An isolated vector comprising the isolated nucleic acid of claim 1.

42. The isolated vector of claim 41, wherein the vector is selected from a PiggyBac (PB) plasmid, Sleep Beauty (SB) plasmid, or a Tol2 plasmid.

43. A host cell comprising the isolated vector of claim 42.

44. A kit comprising:
(a). an isolated nucleic acid of claim 1;
(b) a regulatory molecule for controlling expression of any one of (a), (b), (c), (d), or (e) of the isolated nucleic acid; and
(c) instructions for use.

45. The kit of claim 44, wherein the regulatory molecule is selected from cumate, shield 1, trimethoprim (TMP), tetracycline, doxycycline, arabinose, isopropyl β-D-1-thiogalactopyranoside (IPTG), abscisic acid, gibberellin acid, and/or rapamycin.
